(19) United States
(12) Patent Application Publication
(10) Pub. No.: US 2025/0078041 A1
(43) Pub. Date: Mar. 6, 2025

(54) SYSTEMS AND METHODS FOR MANAGING REPAIRS

(71) Applicant: Viskoot LLC, Mclean, VA (US)

(72) Inventors: Hamid Reza SHAFIE, Mclean, VA (US); Haobo CHENG, Arlington, VA (US); Nathan Junyao ZHU, Mclean, VA (US); Ruitao ZHANG, Arlington, VA (US); Henry DUONG, Aldie, VA (US)

(21) Appl. No.: 18/812,478

(22) Filed: Aug. 22, 2024

Related U.S. Application Data
(60) Provisional application No. 63/535,461, filed on Aug. 30, 2023.

(51) Int. Cl. G06Q 10/20; G06K 19/06; G06Q 30/0601; G06T 7/00

(52) U.S. Cl. CPC G06Q 10/20; G06Q 30/0631; G06T 7/0002; G06K 19/06037

(57) ABSTRACT

The repair systems and methods provide an application platform that has a downloadable application (UI) for mobile devices and a back-end dashboard for the manufacturers for assisting with repairs. The application program streamlines the entire repair journey from when an end-user needs to initiate a repair request until the repaired and re-certified device is shipped back by the manufacturer or repair facility to the end-user. The application may also provide end-users with the option to replace an unrepairable device or to perform a device swap.

### CROSS-REFERENCE TO RELATED APPLICATIONS

This application claims priority to U.S. Provisional Application Ser. No. 63/535,461, filed Aug. 30, 2023, the entire contents of which are hereby incorporated by reference in their entirety.

### FIELD OF THE INVENTION

The present invention relates to systems and methods for managing end-users' experience, real-time tracking of the entire repair journey of a medical device and exchange of information with cloud-based processing systems, diagnosing medical device conditions, and providing recommendations utilizing artificial intelligence (AI). The present invention also provides an interface for medical device manufacturer and repair centers to receive and process the repairs from end-users.

In other embodiments, the present invention relates to systems and methods for managing end-users' experience, real-time tracking of the entire repair journey of power tool and exchange of information with cloud-based processing systems, diagnosing power tool conditions, and providing recommendations utilizing AI. The present invention also provides an interface for power tool manufacturers and repair centers to receive and process the repairs from end-users.

### BACKGROUND

Medical device repair is a totally offline process that leaves the end-user in the dark when the repair journey starts. In today's digital economy, real-time tracking and managing the relationship between end-users and the manufacturers is no longer optional. However, there are currently no systems that interface end-users directly with manufacturers or repair centers. Difficulty in obtaining repairs or replacements may lead to low engagement or respect for the brand Therefore, a need exists for a system that makes the repair journey for medical devices more transparent and enhances the end-user's experience when their medical device is out of order.

Similarly, Power tool repair is a totally offline process that leaves the end-user in the dark when the repair journey starts. In today's digital economy, real-time tracking and managing the relationship between end-users and manufacturers is no longer optional. However, there are currently no systems that interface end-users directly with manufacturers or repair centers. Difficulty in obtaining repairs or replacements may lead to low engagement or respect for the brand Therefore, a need exists for a system that makes the repair journey for the Power tool more transparent and enhances the end-user's experience when their Power tool is out of order.

### SUMMARY

It is an object of the present invention to provide an application platform that has a downloadable application (UI) for mobile devices and a back-end dashboard for the medical device manufacturer. The application platform streamlines the entire repair journey from when a medical device end-user needs to initiate a repair request until the repaired and re-certified medical device is shipped back by the manufacturer or repair facility to the end-user.

In a preferred embodiment, the mobile application back end comprises an AI system for diagnosing and troubleshooting error messages displayed on the medical device display panel without requiring the end-user to manually search for the information. The mobile application may offer troubleshooting instructions or suggest that repair may be needed. If repair is required, the mobile application gathers all the required information from the end-user, such as manufacturer and the size of the medical device. For small-sized medical devices, the mobile application also generates a shipping label for the end-user for shipping the medical device to the correct facility. For medium-sized devices which require a pickup, the mobile automatically will schedule a pickup date and time for the end-user. For large devices which require onsite repair, the mobile application automatically will schedule an onsite repair by a certified technician. For large-sized devices which require the technician from the manufacture/third party repair facility come on-site to repair the unit.

The mobile application provides the end-user with real-time updates for all steps of the repair journey. The mobile application real-time notifications and/or other communication to notify the end-user about changes in the status of the repair journey and possible required actions by the end-user such as authorizing a repair or to discard a broken and unrepairable product. After repair is complete, the mobile application also generates an electronic invoice and the end-user can pay for the repair and return shipping directly through the mobile application.

If the medical device cannot be repaired, the mobile application makes in-application recommendations to buy a brand new identical medical device or the newer generation as a replacement. End-users may also refuse any proposed repairs and request the broken device to be returned or discarded. The back-end of the mobile application automatically syncs with the mobile application and collected NPS (Net Promoter Score) to prioritize the repair timeline of newly arrived medical devices.

In other embodiments, it is an object of the present invention to provide an application platform that has a downloadable application (UI) for mobile devices and a back-end dashboard for the Power tool manufacturer. The application platform streamlines the entire repair journey from when a Power tool end-user needs to initiate a repair request until the repaired and re-certified Power tool is shipped back by the manufacturer or repair facility to the end-user.

The mobile application may offer troubleshooting instructions or suggest that repair may be needed. If repair is required, the mobile application gathers all the required information from the end-user, such as the manufacturer of the Power tool. The mobile application generates a shipping label for the end-user for shipping the Power tool to the correct facility.

The mobile application provides the end-user with real-time updates for all steps of the repair journey. The mobile application real-time notifications and/or other communication to notify the end-user about changes in the status of the repair journey and possible required actions by the end-user such as authorizing a repair or discarding a broken and unrepairable product. After repair is complete, the mobile application also generates an electronic invoice and the end-user can pay for the repair and return shipping directly through the mobile application.

If the Power tool cannot be repaired, the mobile application makes in-application recommendations to buy a brand-new identical Power tool or the newer generation as a replacement. End-users may also refuse any proposed repairs and request the broken device to be returned or discarded. The back-end of the mobile application automatically syncs with the mobile application and collects NPS to prioritize the repair timeline of the newly arrived Power tool.

### BRIEF DESCRIPTION OF THE DRAWINGS

FIG. 1 depicts a network diagram of the repair management system according to an embodiment of the invention.

FIG. 2 depicts a flowchart showing the seps utilized to determine error codes according to an embodiment of the invention.

FIG. 3 depicts a scanner for a mobile application according to an embodiment of the invention.

FIG. 4 depicts an image of a medical device with a displayed error code according to an embodiment of the invention.

FIG. 5 depicts an example error message for troubleshooting according to an embodiment of the invention.

FIG. 6 depicts a flowchart showing the capabilities and features of a mobile application according to an embodiment of the invention.

FIG. 7 depicts an example dashboard for the mobile application according to an embodiment of the invention.

FIG. 8 depicts an example select product type screen according to an embodiment of the invention.

FIG. 9 depicts an example product selections size screen according to an embodiment of the invention.

FIG. 10 depicts an example product information screen according to an embodiment of the invention.

FIG. 11 depicts an example shipping option screen according to an embodiment of the invention.

FIG. 12 depicts an example survey screen according to an embodiment of the invention.

FIG. 13 depicts an example submission screen according to an embodiment of the invention.

FIG. 14 depicts a repair order added to the dashboard according to an embodiment of the invention.

FIG. 15 depicts an example milestone screen according to an embodiment of the invention.

FIG. 16 depicts an example repair report screen according to an embodiment of the invention.

FIG. 17 depicts an example repair exchange program screen according to an embodiment of the invention.

FIG. 18 depicts an example screen showing a repair is not possible according to an embodiment of the invention.

FIG. 19 depicts an example financial transaction screen according to an embodiment of the invention.

FIG. 20 depicts an example listing of repairs according to an embodiment of the invention.

FIG. 21 depicts the primary components of the manufacturer portal according to an embodiment of the invention.

FIG. 22 depicts an example monitor board in accordance with an embodiment of the invention.

FIG. 23 depicts an example receive shipment panel according to an embodiment of the invention.

FIG. 24 depicts an example status screen according to an embodiment of the invention.

FIG. 25 depicts an example scan screen according to an embodiment of the invention.

FIG. 26 depicts example decoded information screen according to an embodiment of the invention.

FIG. 27 depicts an example supervisor panel according to an embodiment of the invention.

FIG. 28 depicts a pop-up window according to an embodiment of the invention.

FIG. 29 depicts an example technician panel according to an embodiment of the invention.

FIG. 30 depicts an example repair update screen according to an embodiment of the invention.

FIG. 31 depicts an example product recommendation flowchart according to an embodiment of the invention.

FIG. 32 depicts a network diagram of the repair management system according to an embodiment of the invention.

FIG. 33 depicts a flowchart showing the capabilities and features of a mobile application according to an embodiment of the invention.

FIG. 34 depicts an example dashboard for the mobile application according to an embodiment of the invention.

FIG. 35 depicts an example select product type screen according to an embodiment of the invention.

FIG. 36 depicts an example product information screen according to an embodiment of the invention.

FIG. 37 depicts an example shipping option screen according to an embodiment of the invention.

FIG. 38 depicts an example submission screen according to an embodiment of the invention.

FIG. 39 depicts a repair order added to the dashboard according to an embodiment of the invention.

FIG. 40 depicts an example milestone screen according to an embodiment of the invention.

FIG. 41 depicts an example repair report screen according to an embodiment of the invention.

FIG. 42 depicts an example repair exchange program screen according to an embodiment of the invention.

FIG. 43 depicts an example screen showing a repair is not possible according to an embodiment of the invention.

FIG. 44 depicts a product recommendation screen according to an embodiment of the invention.

FIG. 45 depicts an example listing of repairs according to an embodiment of the invention.

FIG. 46 depicts the primary components of the manufacturer portal according to an embodiment of the invention.

FIG. 47 depicts an example monitor board in accordance with an embodiment of the invention.

FIG. 48 depicts an example receive shipment panel according to an embodiment of the invention.

FIG. 49 depicts an example status screen according to an embodiment of the invention.

FIG. 50 depicts an example scan screen according to an embodiment of the invention.

FIG. 51 depicts example decoded information screen according to an embodiment of the invention.

FIG. 52 depicts an example supervisor panel according to an embodiment of the invention.

FIG. 53 depicts a pop-up window according to an embodiment of the invention.

FIG. 54 depicts an example technician panel according to an embodiment of the invention.

FIG. 55 depicts an example repair update screen according to an embodiment of the invention.

FIG. 56 depicts an example product recommendation flowchart according to an embodiment of the invention.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter as defined in the accompanying claims.

### Medical Device Repair

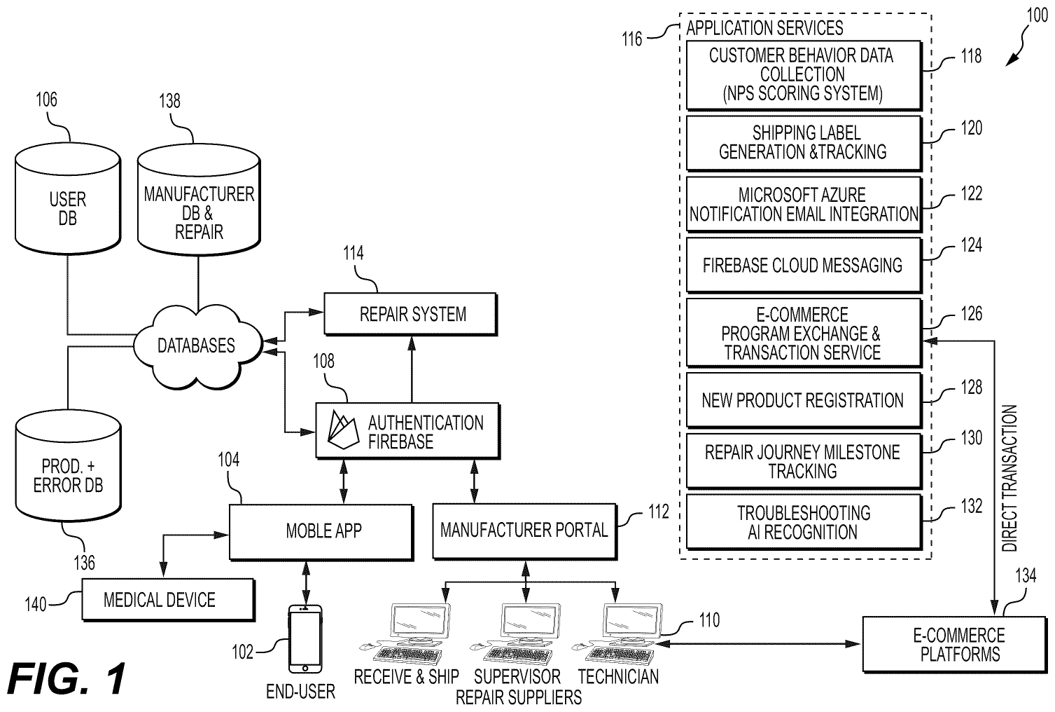

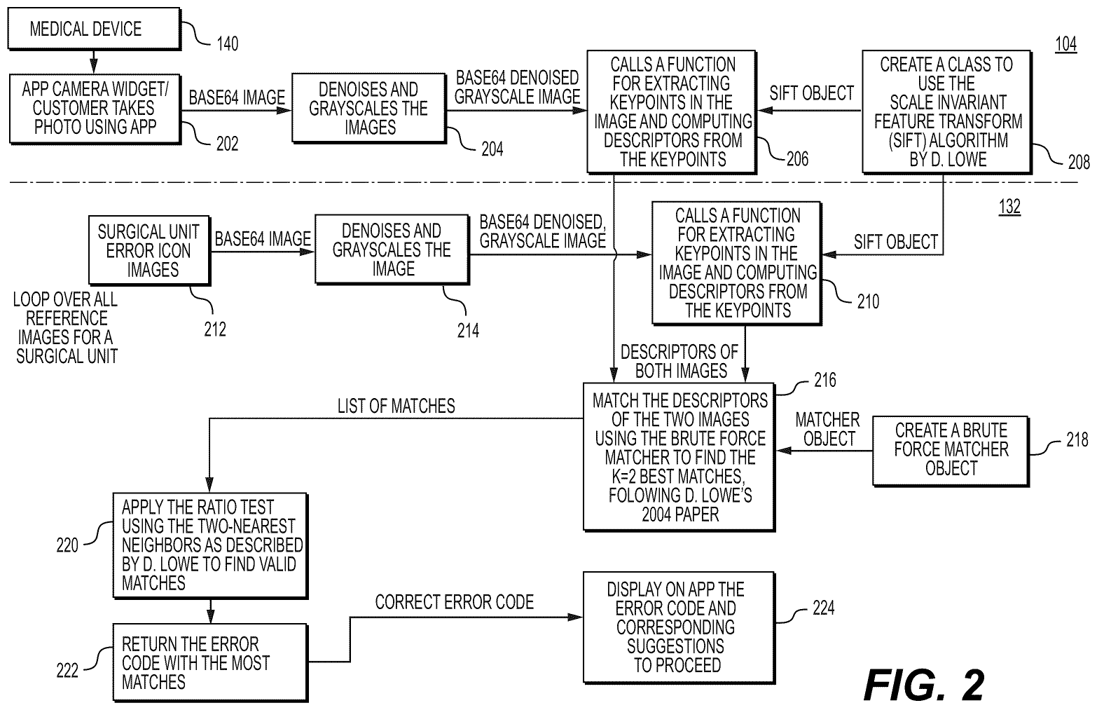

Referring first to FIG. 1, depicted is a network diagram of repair management system 100 showing the connection between end-users, manufacturers, and repair technicians through the mobile application and back-end. End-users 102 can access mobile application 104 by downloading a dedicated mobile application to a smartphone or tablet. Alternatively, the end-user can access a web-app through a browser that provides similar functionality to the mobile application 104. In order to use mobile application 104, each end-user 102 must register with repair management system 100. Identifying information such as name, company, address, etc. is collected from each end-user 102 and is stored in user database 106. This allows all repairs received within repair management system 100 to be associated with a particular end-user 102.

In a preferred embodiment, firebase authentication service 108 is utilized because it supports authentication using passwords, phone numbers, and log-in through popular identity providers like Google, Facebook, Twitter, etc. Firebase authentication service 108 can also support other forms of authentication such as fingerprint identification, multi-factor authentication, or facial recognition. It should be apparent to one of ordinary skill in the art that any secure authentication system may be utilized.

In some embodiments, firebase authentication service 108 is utilized to authenticate both end-users 102 and repair suppliers 110. However, separate systems may be utilized if different levels of authentication are needed.

Repair suppliers 110 may be third-party repair technicians or manufacturers. Access to repair management system 100 for repair suppliers 110 can be accessed through manufacturer portal 112 using a dedicated mobile application (like with end-users 102) or a web application provided through a browser.

Communication and interaction between mobile application 104 and manufacturer portal 112 is managed by repair system 114. Further, repair system 114 also controls the various application services 116 used to manage repair management system 100. Application services 116 may include, but are not limited to, customer data collection system 118, shipping label generator & tracker 120, notification system 122, messaging system 124, financial transaction system 126, product registration system 128, repair update and tracking system 130, and AI module 132.

In some embodiments, the mobile application 104 may suggest new or replacement items to end-users 102 if it is determined that repairs cannot be completed. For example, manufacturer database 138 can store a list of related or suitable replacement items in association with each power tool. End-users 102 can utilize e-Commerce platforms 134 to purchase any needed medical devices directly from the manufacturer.

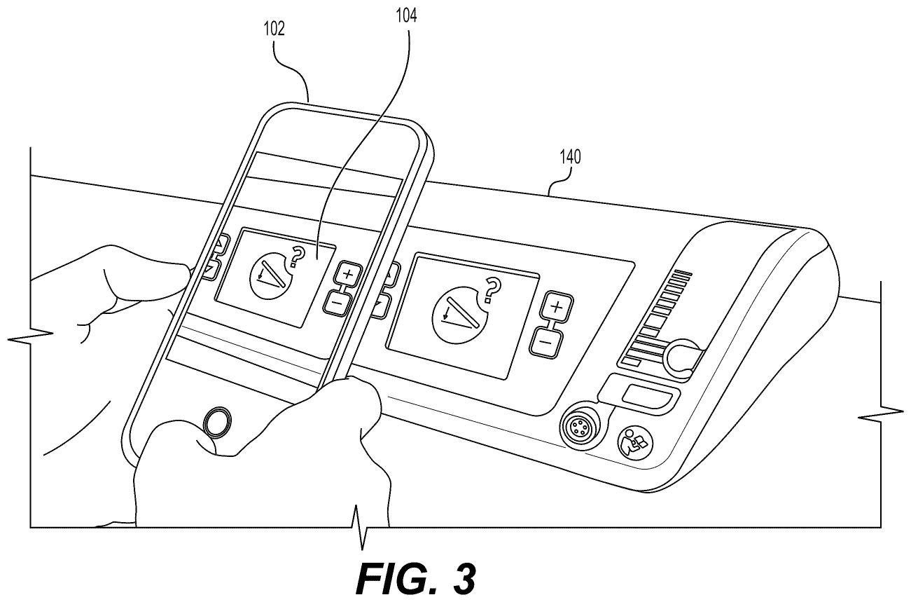

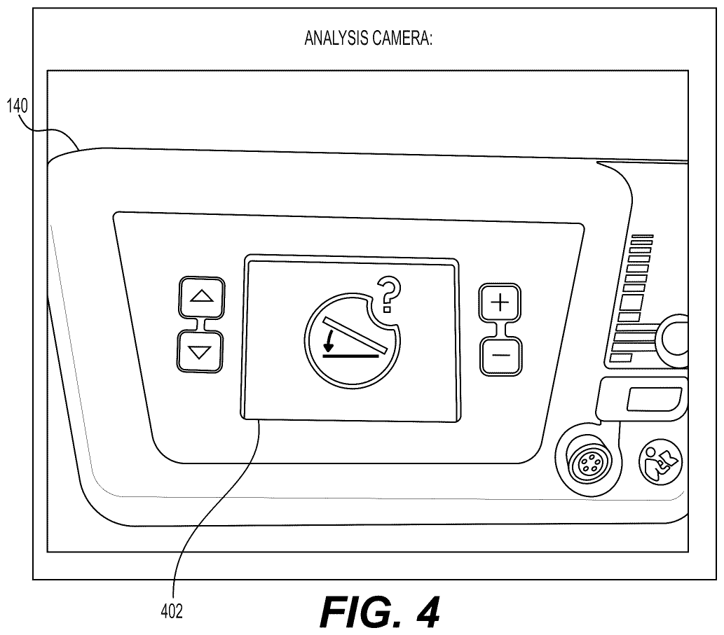

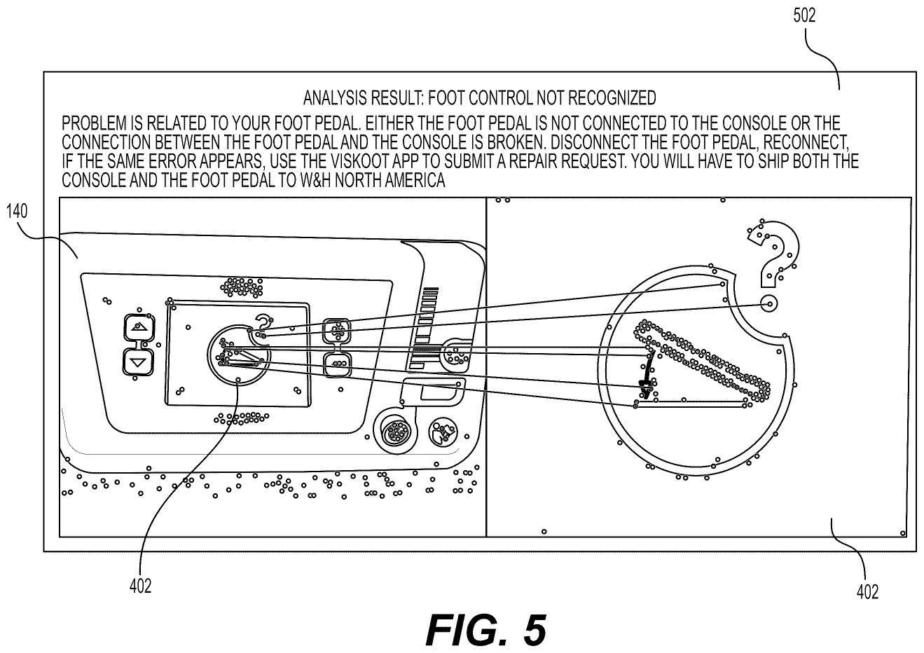

As will be discussed in greater detail later, AI module 132 is able to detect error messages and provide troubleshooting for medical devices 140. The end-users 102 use mobile application 104 to image the medical device 140 displaying the error and AI module 132 identifies the error message, medical device 140, and/or manufacturer and automatically suggests troubleshooting steps to the end-user 102 or suggests the end-user 102 initiate a repair if the medical device 140 requires repair or replacement. The end-user 102 then follows the procedures discussed with reference to FIGS. 3-4. Successful scans will result in the end-user 102 being provided with troubleshooting instructions regarding any detected error codes as shown in FIG. 5.

depicts the steps utilized by AI module 132 to determine error codes for medical devices 140. First, the end-user 102 activates the scanning option on the mobile application 104 in step 202 as depicted in FIG. 3 and uses the live view feed to image the medical device 140 or the error code. An example image showing a medical device error code 402 is depicted in FIG. 4. The end-user 102 may be provided with written or audio instructions regarding the placement of the camera. For example, if the error message 402 is off-center, the end-user 102 may be prompted to center the error message 402 in the camera's field of view. Before passing the collected images to AI module 132 for recognition, the mobile application 104 first processes the images to achieve better detection results. The images are denoised and grayscaled in step 204. This improves the image quality and helps to make the gathered images uniform. The mobile application 104 then identifies keypoints in the images in step 206 and computes descriptors from the keypoints.

The mobile application 104 also creates a class to use a scale invariant feature transform (SIFT) algorithm on the images in step 208. For example, the mobile application may utilize the SIFT algorithm described in Lowe, D. “Distinctive image features from scaleinvariant keypoints” International Journal of Computer Vision, 60, 2 (2004), pp. 91-110, the contents of which are hereby incorporated by reference in their entirety. It should be obvious that other SIFT techniques (e.g., Fast approximated SIFT) may alternatively be utilized.

The resulting SIFT objects are passed to the AI module which calls a function to extract keypoints and compute descriptors using the SIFT objects in step 210. The keypoints may be identified from a plurality of error message images (encoded in Base64) (step 212) which have been denoised and grayscaled in step 214.

The descriptors computed in steps 206 and 210 are matched in step 216 using a brute force method to find the k=2 best matches. The brute force method matcher object used in the brute force matching are computed in step 218. A ratio test is then applied to the two-nearest neighbors detected in step 216 and the error code 402 with the most matches is returned in step 222. The resulting error code is displayed on the mobile application 104 along with a description of the error code and any troubleshooting steps that the end-user 102 may take in step 224. For example, FIG. 5 depicts that the error message has been identified as “foot control not recognized” and provides more description and troubleshooting steps 502 that may be used to correct the error.

The AI module 132 employs a machine-learning model specifically designed for pattern recognition within images. To train this model effectively, a substantial number of product images displaying error signs on medical devices 140 are gathered and serve as the training set. Once the model is thoroughly trained, it becomes adept at identifying the error signs in our product images.

Each detected error message 402 is associated troubleshooting text that is stored in error database 136. For each error, the troubleshooting text 502 may be the same as that contained in the user manual or provided online. However, the end-user 102 does not have to search for the error-message because it is automatically detected by AI module 132. The troubleshooting text 502 may be supplemented by the manufacturer. This gives the manufacturer a centralized location to update troubleshooting steps which can then be disseminated to end-users 102 through mobile application 104.

### Mobile Application 104 and Interface

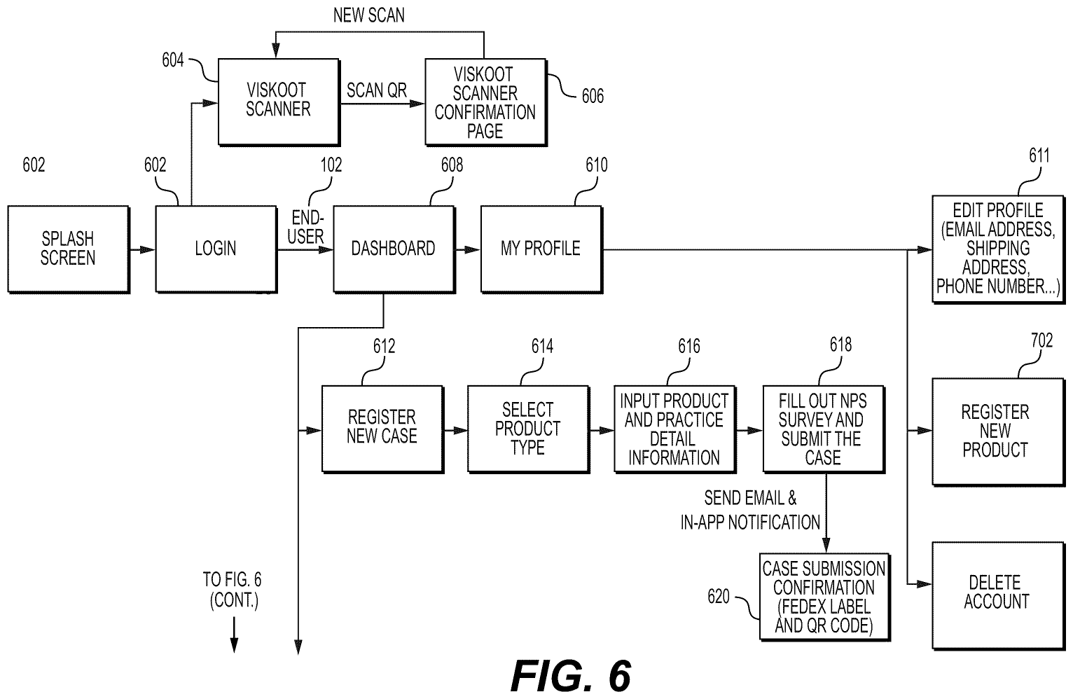

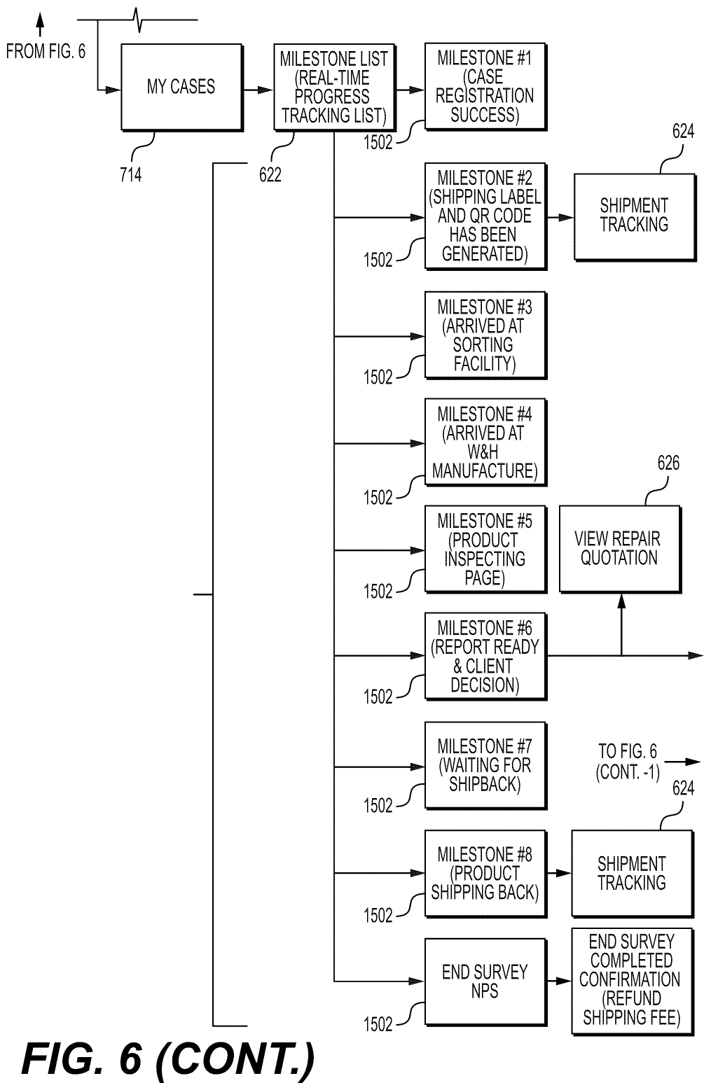

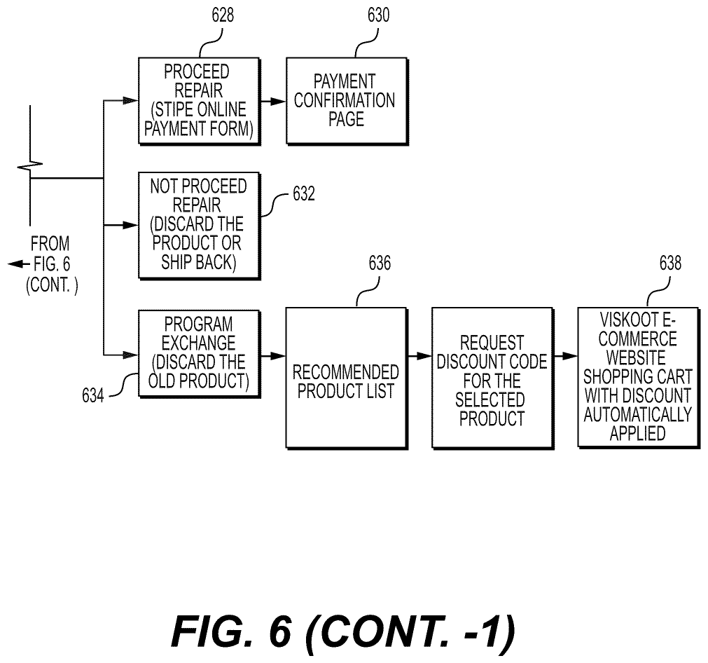

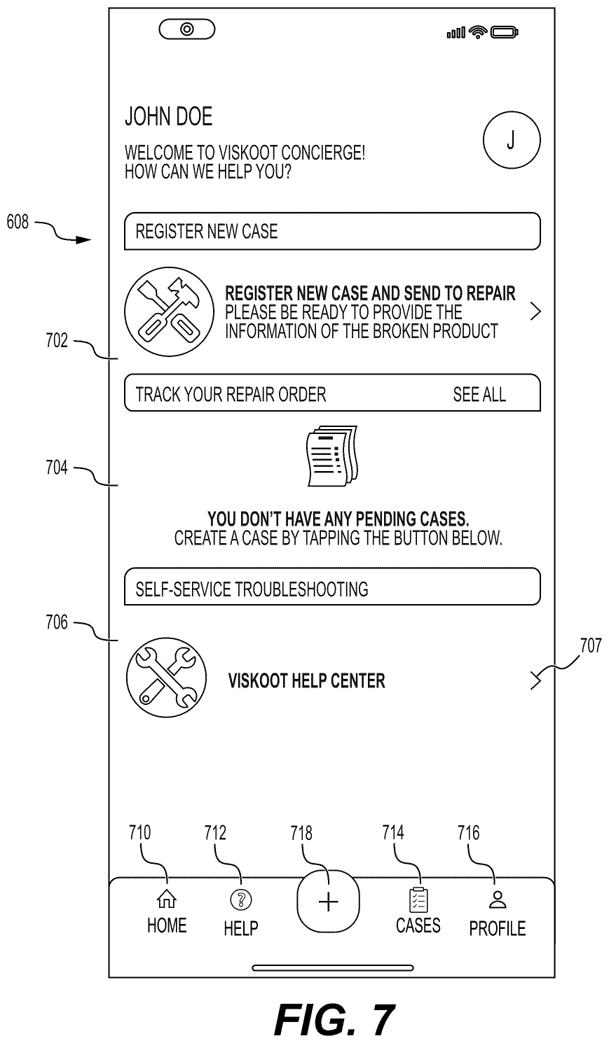

FIG. 6 depicts a flowchart showing the capabilities and features of mobile application 104. The mobile application 104 allows the end-user 102 to login in step 602 and be authenticated through Firebase authentication service 108. After an end-user 102 is authenticated in step 602, a dashboard 608 is displayed to the end-user 102 as depicted in FIG. 7. In a preferred embodiment, the dashboard 608 is displayed to every end-user 102 after being authenticated. The dashboard 608 includes sections for registering new products 702 that can be used to register any medical devices in case later repair requests are needed. This allows end-users 102 to provide the medical device information one time and it is saved in association with the end-user information in user database 106. The tracking section 704 provides the end-user 102 with a listing of all active repair requests. If additional help is needed with the mobile application 104, the end-user 102 can select help center selection 706 to be directed to a help center 707. The help center 707 may include frequently asked questions (FAQ) to provide the most common answers or additional options like the ability to chat or call a live agent to discuss the issue.

In some embodiments, the bottom of dashboard 608 includes access menu 708 which is static and is displayed on most pages of mobile application 104. Access menu 708 may provide a home option 710 that can be used to return to dashboard 608 at any point, a help option 712 to access additional help related to the mobile application 104, a cases option 714 allowing the end-user 102 to view all active and closed repair requests, and a profile option 716. If the end-user 102 selects the profile option 716, a my profile screen 610 is displayed listing the profile information saved in mobile application 104 in user database 106. The end-user 102 can select any of the displayed fields such as e-mail address, address, shipping address, phone number, etc. in step 611 to provide new or update profile information. After any changes are made to the information on the my profile screen 610, the user database 106 is updated to reflect the changes. The dashboard also comprises a plus option 718. The plus option 718 can be used to initiate a repair request without the end-user 102 having to return to the dashboard 608.

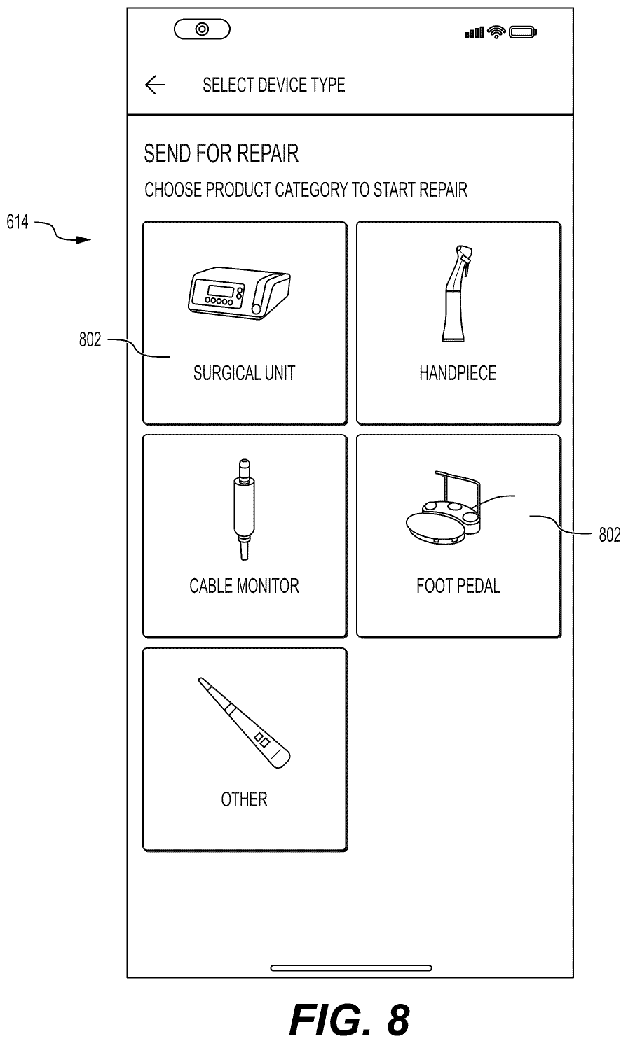

If the end-user 102 selects the plus option 718, a new repair request is initiated in step 612 so that all necessary information regarding the repair request can be collected. In a preferred embodiment, a select product type screen 614 is first displayed to the end-user 102 so that a category can be selected as depicted in FIG. 8. Selection options 802 may include surgical unit, hand piece, cable motor, foot pedal, or other.

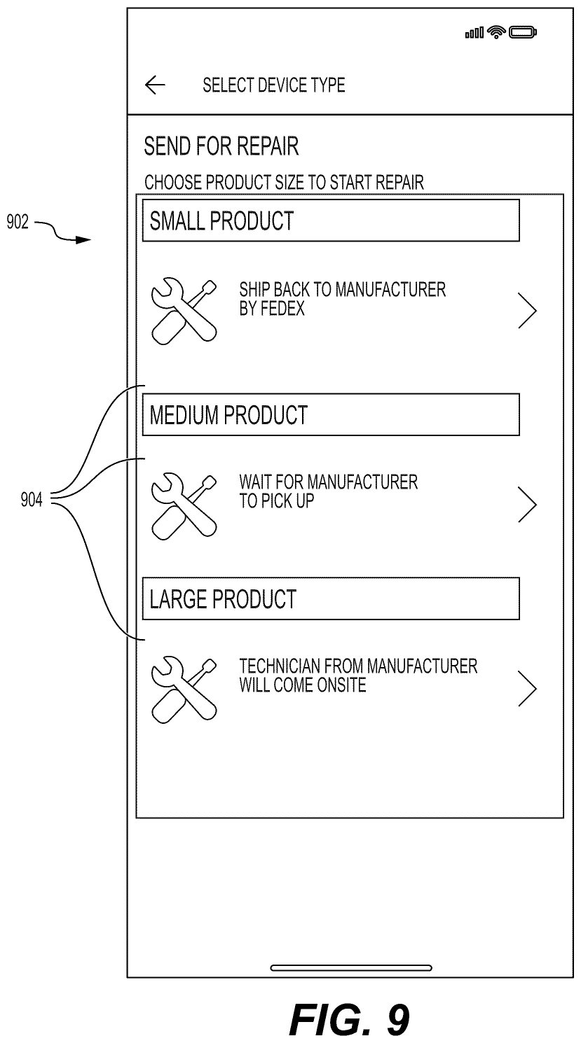

In some embodiments, the end-user 102 is first directed to select a product size on product size screen 902 as depicted in FIG. 9. Selection options 904 may include small product, medium product, or large product. Generally, a small product is a medical device 140 that can be shipped using standard shipping methods such as FedEx, USPS, UPS, etc. Medium products are medical devices 140 that can be shipped but would require an on-site pickup due to the dimensions or weight. Other shipping options, such as LTL shipping, may be required for medium products. Large products are generally medical devices 140 that cannot be shipped due to their delicacy, size, or if they are installed (e.g., a large surgical unit). After end-user 102 selects product size, end-user 102 is directed to product type screen 614. Alternatively, a product size may be associated with each product listed in product/error database 136 and the appropriate product size can automatically be determined after the exact medical device is known.

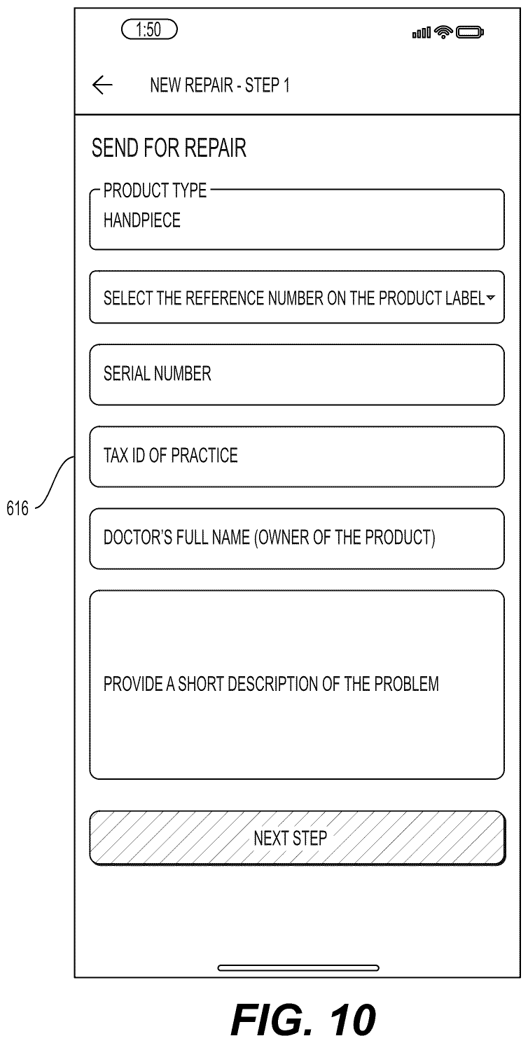

The end-user 102 is then prompted to enter additional product information on product information screen 616. An example product information screen 616 is depicted in FIG. 10. The additional product information may the product's reference number, serial number, tax ID, the name of the owner or operator of the product as well as a short description of the repair issue.

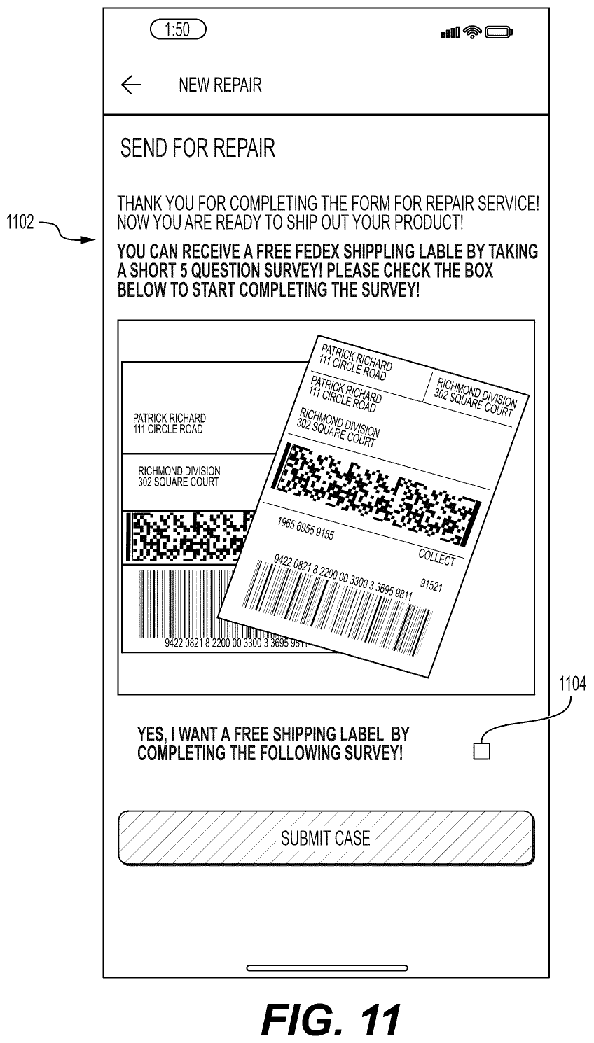

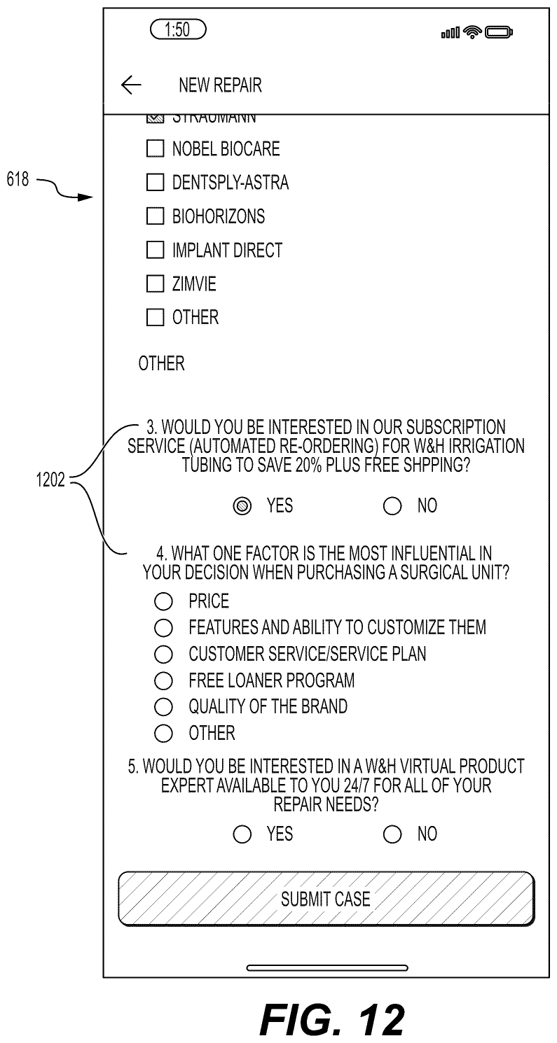

If the medical device 140 is a small product that can be shipped, the end-user 102 may be provided with a shipping option screen 1102 as depicted in FIG. 11 informing them that they can received free shipping by filling out a survey. If the end-user 102 selects the check box 1104, the mobile application 104 displays survey screen 618. An example survey screen 618 generated by customer data collection system 118 is depicted in FIG. 12 listing questions 1202.

In a preferred embodiment, each product stored in error and product database 136 is associated with a particular repair location for that item. The repair location may be a third-party repair service or the manufacturer's own repair service. The manufacturer can select their preferred repair location for each of their products. For example, a manufacturer may choose to have products of a first type be repaired by a particular third-party repair shop whereas products of a second type are shipped directly to the manufacturer. Because the repair location information is stored ahead for each medical device, the repair location address can automatically be pulled by repair management system 100. The end-user 102 does not have to determine the address. The end-user is only required to affix a generated shipping label to the medical device packaging as it already has the correct address specified in advance by the manufacturer.

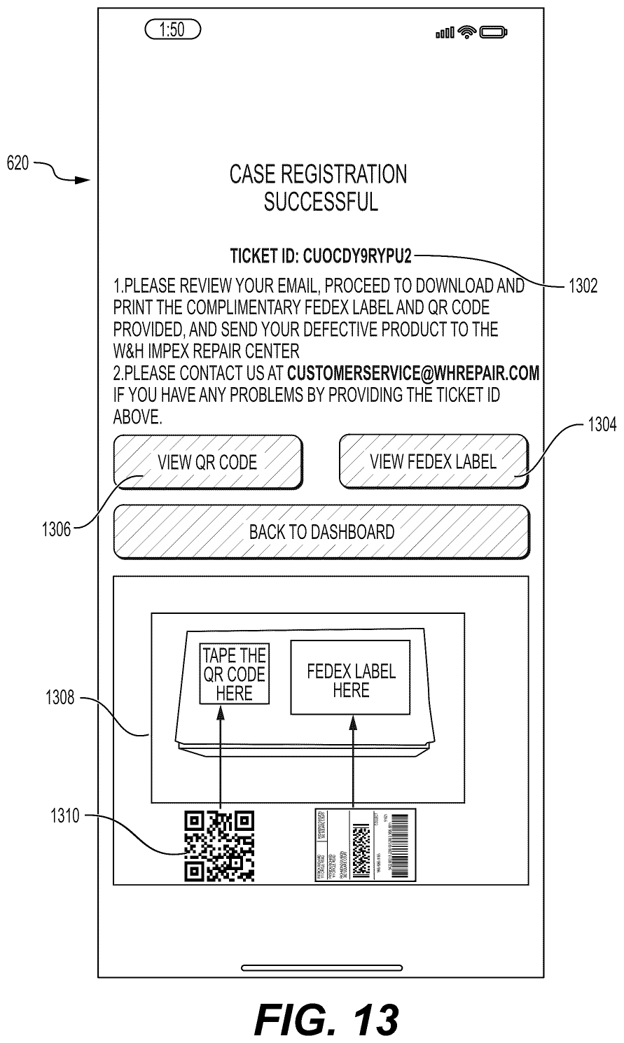

After the end-user 102 completes the survey screen 618, a submission screen 620 is displayed to the end-user 102. An example submission screen 620 is depicted in FIG. 13. The end-user 102 is provided with a confirmation number 1302 which is used to uniquely identify each repair request. The confirmation number 1302 may comprise a combination of alphanumeric characters and numbers as is known in the art. The confirmation number 1302 is stored in association with all of the repair request information gathered. The tracking number for the shipping label is also stored in association with the confirmation number 1302. Selecting shipping label option 1304 prompts shipping label generator and tracker 120 to produce a shipping label for the end-user 102.

A machine-readable code 1310 is also generated by selecting code button 1306. In a preferred embodiment, the machine-readable code 1310 that is generated encodes information related to the repair such as confirmation number 1302, the tracking number, the address, etc. The amount of information that can be included in the machine-readable code 1310 is only limited by the capabilities of the type of machine-readable code. For example, a ID barcode can generally only include up to 85 encoded characters whereas a 2D barcode, such as a QR code, can encode 3000+ characters if needed. The encoded information in the QR code may optionally be encrypted if the characters include sensitive personal information such as the shipper's return address. An instruction image or video 1308 may be provided on submission screen 620 showing how the shipping label and machine-readable code should be attached to the packaging.

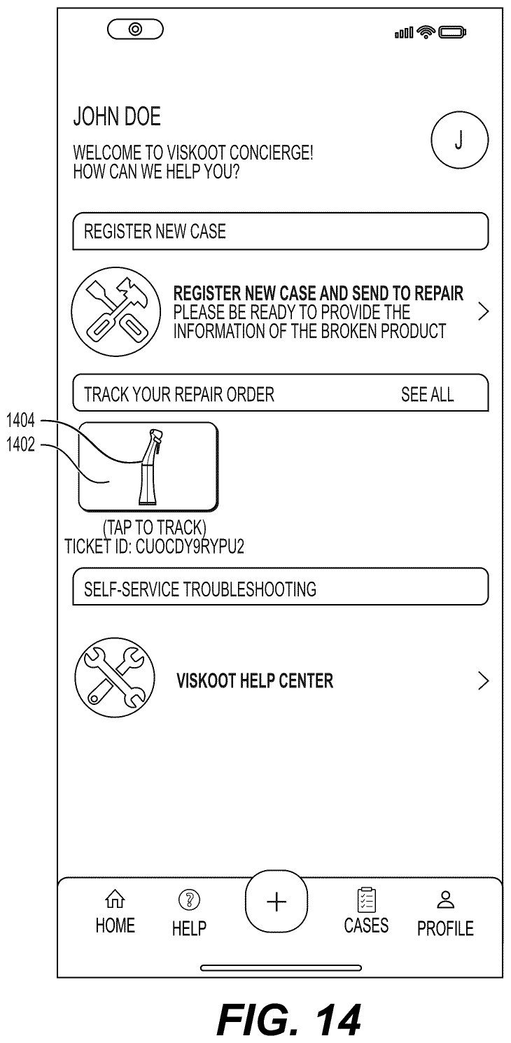

The repair order 1402 is then added to dashboard 608 as depicted in FIG. 14. The repair order 1402 preferably includes an image 1404 of the medical device 140 being repaired and/or the confirmation number 1302. The end-user 102 can select repair order 1402 to view case milestones associated with repair order 1402.

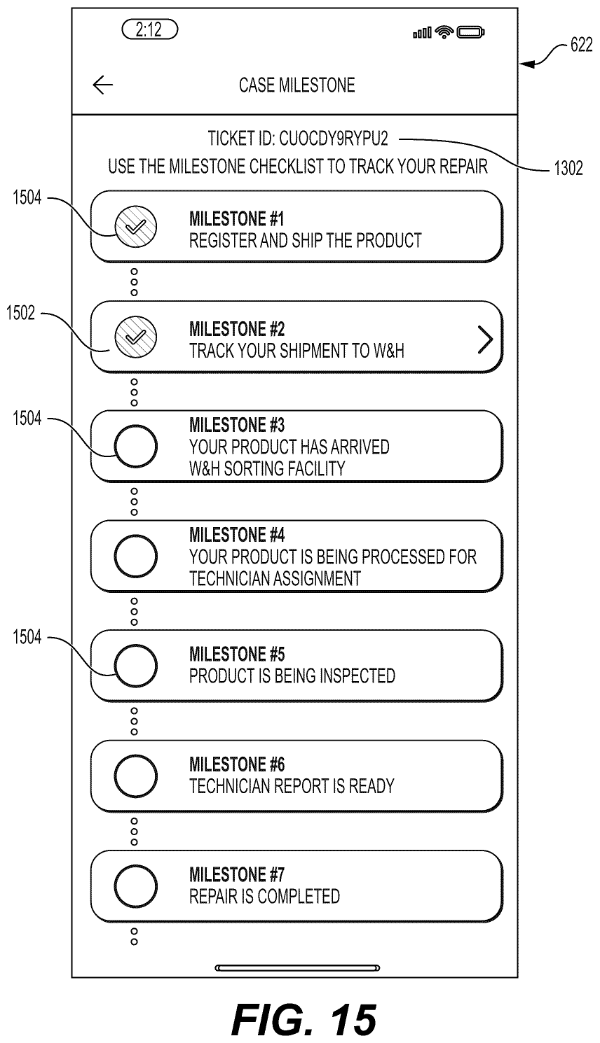

FIG. 15 depicts an example milestone screen 622 showing the real-time status of the repair process for repair order 1402. For example, after the end-user 102 has shipped the medical device 140, second milestone 1502 can be selected by the end-user 102 to view the shipping status. For example, the tracking information associated with the confirmation number 1302 can be used to retrieve the associated tracking number and the end-user 102 can be directed to a screen or website 624 showing the current shipment status (e.g., in transit, arrived, etc.). As each milestone 1502 in the repair process is completed, an icon 1504 associated with the milestone 1502 is changed to reflect completion. For example, check marks can be added to the icons 1504 for each milestone 1502 which allows the end-user 102 to quickly identify the current status through a visual inspection.

As will be discussed later, the additional milestones 1502 receive checkmarks as the medical device 140 proceeds through repair. The confirmation number 1302 is also used by the repair facility 110 to track the repair process. As each repair step is completed and the information is updated, the corresponding milestone 1502 is activated. For example, when a shipment arrives at the repair facility 110, the QR code 1310 is scanned and the status of the case 1402 is changed to “received.” This triggers the icon 1504 for the third milestone 1502 to be changed to a check mark. Various actions, or combinations of actions, performed at the repair facility can be used to activate or deactivate certain milestones 1502. The activation of each milestone 1502 may also cause certain other actions to be performed such as sending a text message, email, etc. The rules for triggering each milestone 1502 are controlled by repair update and tracking system 130 and are configurable by a technician or manufacturer.

No action is required by the end-user 102 until the sixth milestone 1502 is activated which indicates that the repair report is ready. Generally, when the repair report 626 is ready, the end-user 102 will be provided with one or more notifications because the end-user 102 must select a repair option.

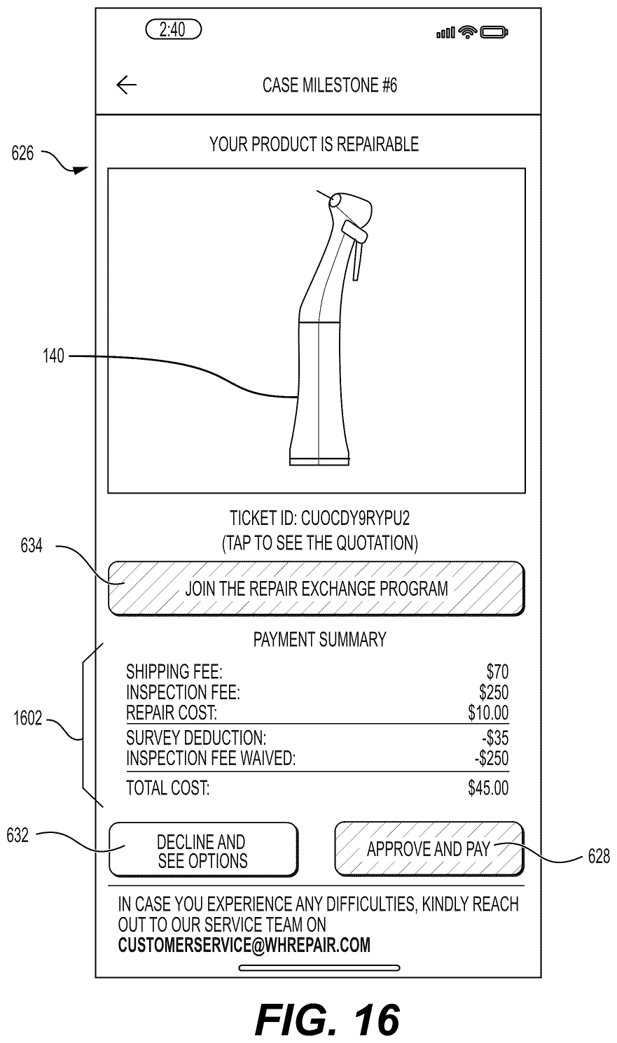

Selection of the sixth milestone 1502 causes a repair report screen 626 to be displayed. Example repair report screens 626 are depicted in FIGS. 16-19. The content of the repair report 626 determines which repair report screen 626 is displayed to the end-user 102. If the medical device 140 can be repaired, the repair report screen 626 displayed in FIG. 16 is displayed. The end-user 102 is informed of the repair cost in payment summary section 1602 which includes itemized costs and reflects and discounts for the end-user 102. If the end-user 102 selects the approve option 628, the end-user 102 can utilize financial transaction system 126 to pay for the repairs. A payment confirmation page 630 is generated informing the end-user 102 that payment is completed and the requested repair will be completed. A payment confirmation 630 is generated after the repair has been approved and paid for. If the repair is no longer required, the end-user 102 can select decline option 632 and choose to have the medical device 104 returned or destroyed.

The end-user can also choose product exchange option 634 which allows the end-user 102 to purchase a similar or replacement medical device at a discount. This option can be used to direct end-users 102 to purchase replacements directly from the manufacturer instead of a third-party without the end-user having to exit the mobile application 104.

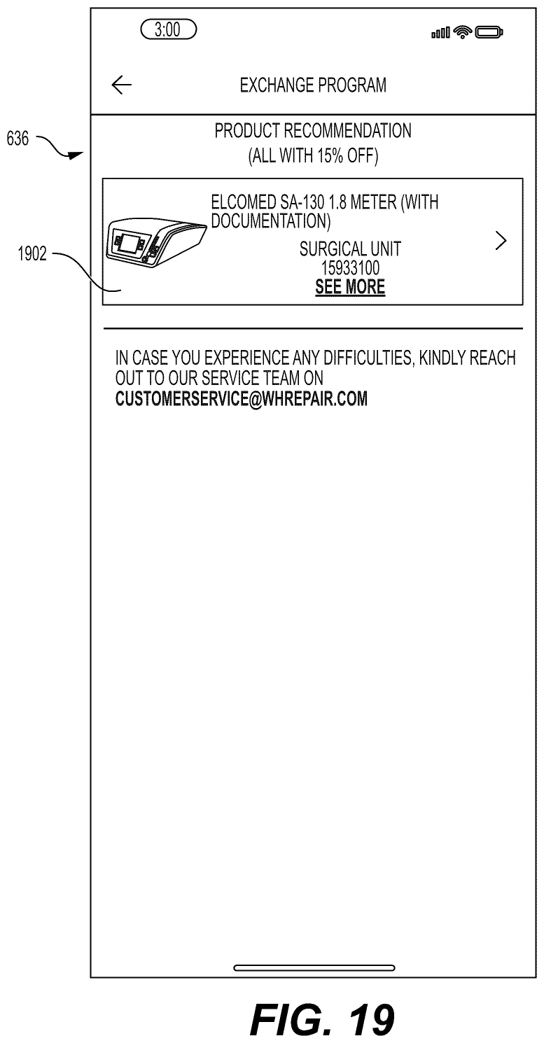

Selecting product exchange option 634 directs the end-user 102 to a product recommendation page 636 listing products that are suitable as a replacement. The end-user 102 can select one of the displayed recommendations 1902 and pay for the purchase using financial transaction system 126 as depicted in FIG. 19 (step 638).

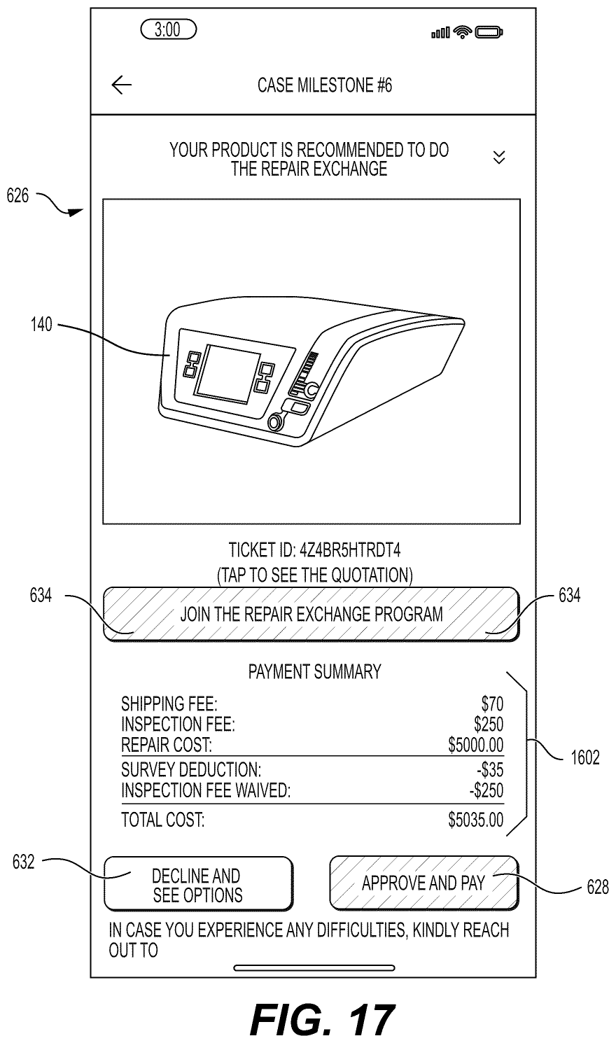

In certain instances, the repair cost may be comparable to or greater than the cost of a completely new or refurbished product. If a replacement cost is within a certain predefined threshold of the repair cost or greater than the repair cost, the repair report screen 626 of FIG. 17 may be displayed to the end-user 102 indicating that it is recommended to join the repair exchange program. However, the end-user 102 is still free to also choose to have the repair performed by selecting approve option 628.

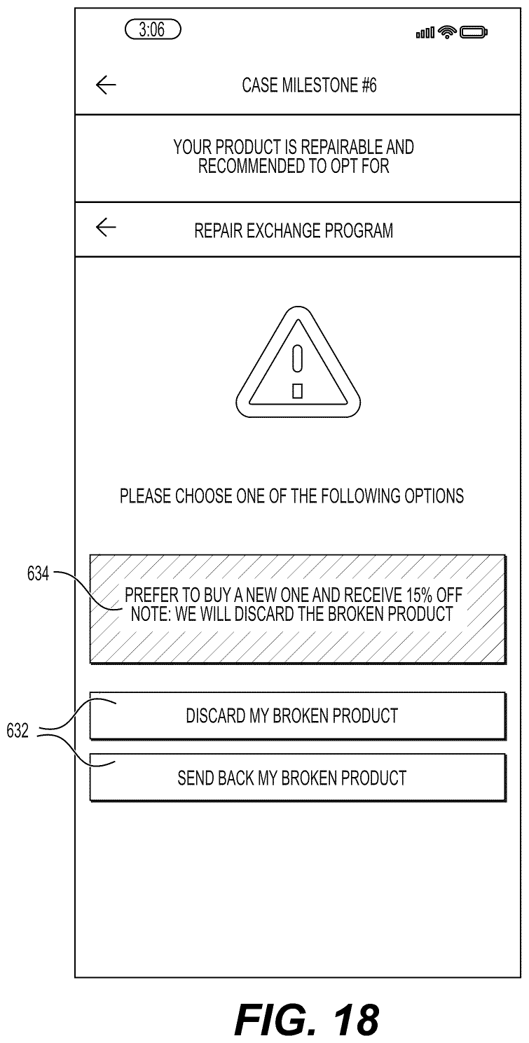

The repair report screen 626 may also indicate that the product is not repairable as depicted in FIG. 18. The end-user 102 can select product exchange option 634 to buy a new product at a discount or select decline option 632 and choose to have the product returned or destroyed. If the end-user 102 chooses to have the object returned, the end-user 102 is prompted to pay for the return shipping by financial transaction system 126.

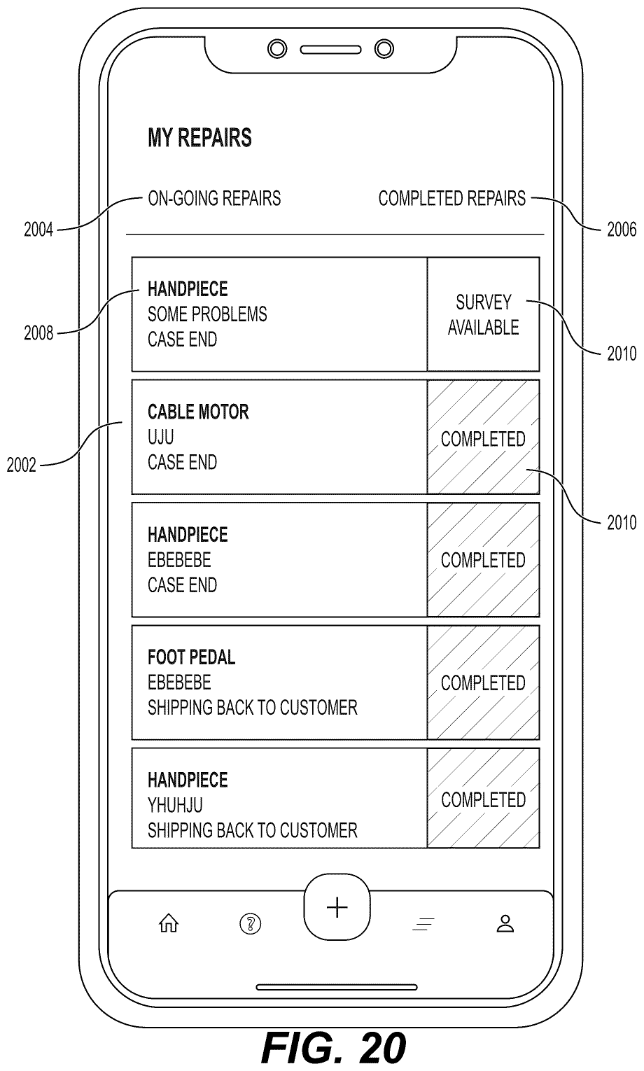

Returning to FIG. 7, if end-user 102 chooses cases option 714 from dashboard 608, a listing 2002 of all repairs associated with the logged-in end-user 102 are displayed as depicted in FIG. 20. The end-user 102 can toggle the display by selecting the on-going repairs option 2004 or the completed repairs option 2006 to active or closed repair requests, respectively. For each repair request, identifying information 2008 is provided adjacent to most recent status 2010. In some embodiments, the most recent status 2010 may indicate that a survey 618 is available to the end-user 102 related to the repair. The end-user 102 can fill out the survey 618 to indicate their experience and satisfaction with the repair. Submitting the survey 618 may result in full refund for return shipping cost of repaired product.

### Manufacturer Portal 112

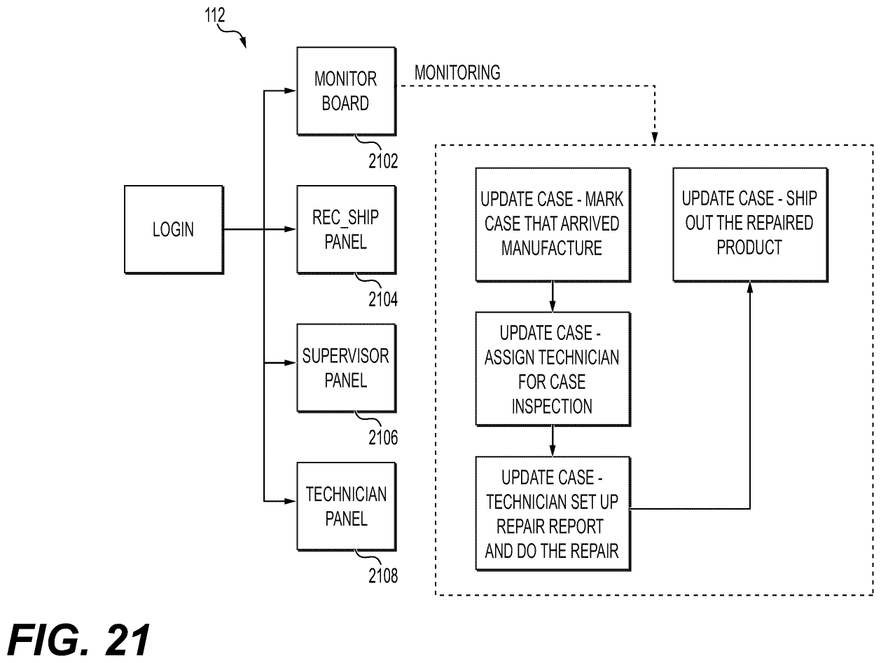

As previously discussed with reference to FIG. 1, each repair supplier 110 has access to manufacturer portal 112 that can be used to scan received repairs, track the status of repairs, and ship out completed repairs. The main elements of manufacturer portal 112 are depicted in FIG. 21. Manufacturer portal 112 generally comprises monitor board 2102, receive shipment panel 2104, supervisor panel 2106, and technician panel 210. Each user of manufacturer portal 112 can be assigned one or more designations or access levels which determine which of the features of manufacturer portal 112 can be accessed. For example, upper management may only be provided access to monitor board 2102 whereas a supervisor may be provided access to both monitor board 2102 and supervisor panel 2106 but restricted from accessing technician panel 2108.

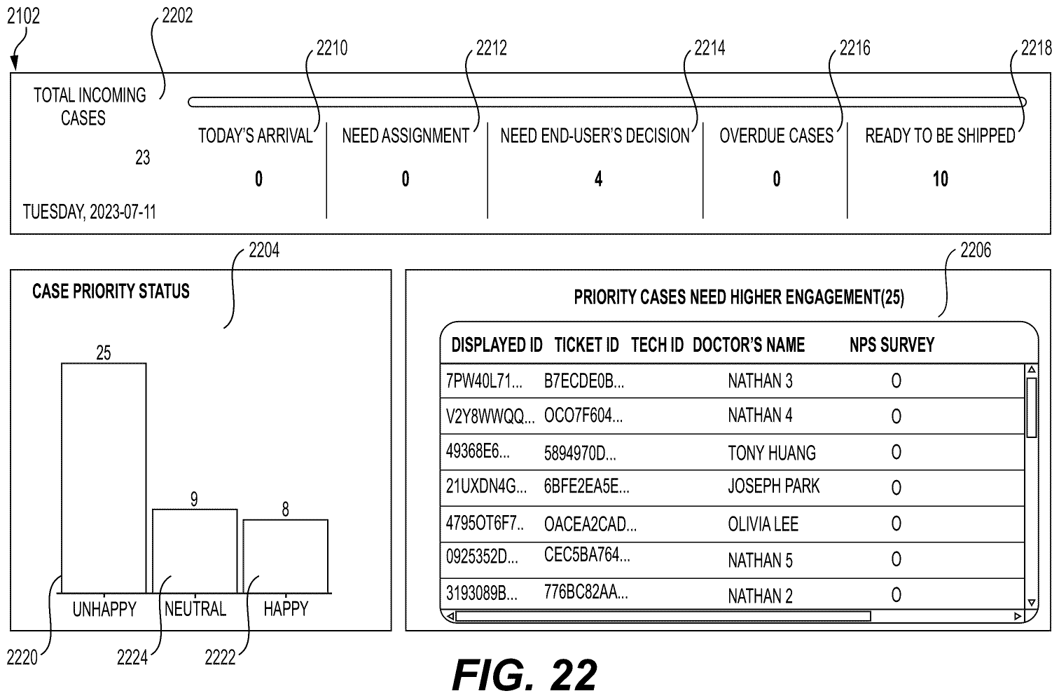

An example monitor board 2102 comprising three sections is depicted in FIG. 22. Monitor board 2102 generally comprises three parts: case number index 2202, case priority status 2204, and priority cases 2206. Case number index 2202 displays statuses related to daily or weekly statistics and may include the following categories:
Total Incoming Cases 2208 indicates the number of repair cases currently being sent to the shipping team.
Today's Arrival 2210 shows the cases that have arrived at the repair supplier 110.
Need Assignment 2212 are the cases that have not been assigned to a technician yet and are now waiting to be assigned by the supervisor.
Waiting for End-user Decision 2214 includes cases where the technician has sent the repair report to the end-user 102 and is awaiting their decision.
Overdue Cases 2216 indicates Cases that have exceeded the estimated repair or inspection time of 24 hours.
Ready to be Shipped 2218 displays cases that have been repaired or returned and are ready to be shipped out by the shipment team.

To help the upper management and the whole team quickly grasp the sentiment of each case, different colors may be used indicate the urgency of each case based on end-users' feedback that they provide during their case submission. Red may be used to represent unhappy end-users 2220 who are disengaged with the medical device brand. These end-users 2220 can be highly prioritized and addressed immediately with a fast turnaround time. Green may be used to represent strongly satisfied end-users 2222 who are fully engaged with the medical device brand. They can have repairs done within the normal turnaround time. Yellow may be used to represent neutral end-users 2224 who indicated neither strongly satisfied nor strongly dissatisfied feedback. They typically receive medium priority for a slightly higher than normal turnaround.

The case priority index 2204 shows the number of repairs falling into each of the different categories. It should be obvious that the number of end-user categories and/or the display method used to represent each category can be varied as needed by each repair supplier 110.

Priority cases 2206 provides a listing of all cases marked red from unhappy end-users 2220. These cases require immediate attention as end-users 2220 are unhappy or have urgent repair needs. Priority cases 2206 may display information including Case ID, assigned technician, doctor's name, NPS survey result, and the current status. By referring to this table, upper management can easily identify these priority cases.

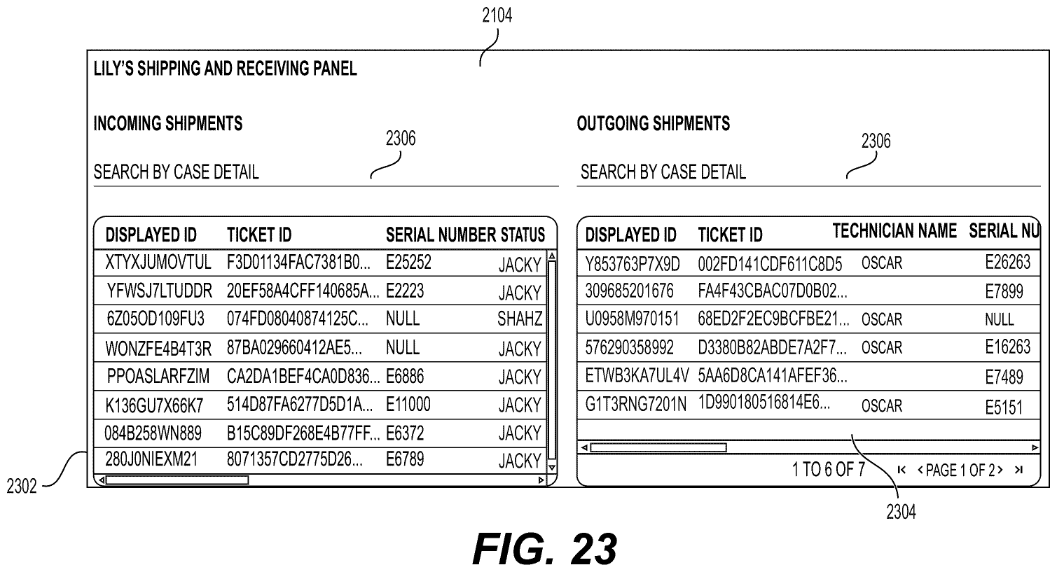

FIG. 23 depicts a sample receive shipment panel 2104. In a preferred embodiment, each member designated as mailroom or shipping has access to receive shipment panel 2104 after being authenticated. The receive shipment panel 2104 is divided into two main sections, specifically designed to provide an overview of both incoming and outgoing repair cases. It also offers powerful search and modification features.

Incoming shipments section 2302 provides an organized list of all incoming cases destined for the repair supplier 110 and displays corresponding statuses. Like in the monitor board 2102, the entries may be color coded according to urgency based on end-users' feedback provided during their case submission. This clear view of the case statuses allows supervisors and technicians to prioritize and allocate resources effectively, ensuring prompt and efficient resolution.

The outgoing shipment section 2304 is similar to the incoming shipment section 2302 but provides information on cases that have been shipped from the repair supplier 110. The incoming shipment section 2302 and outgoing shipment section 2304 also comprise search field 2306 used to locate specific cases by typing any identifying information such as confirmation number 1302, end-user name, etc.

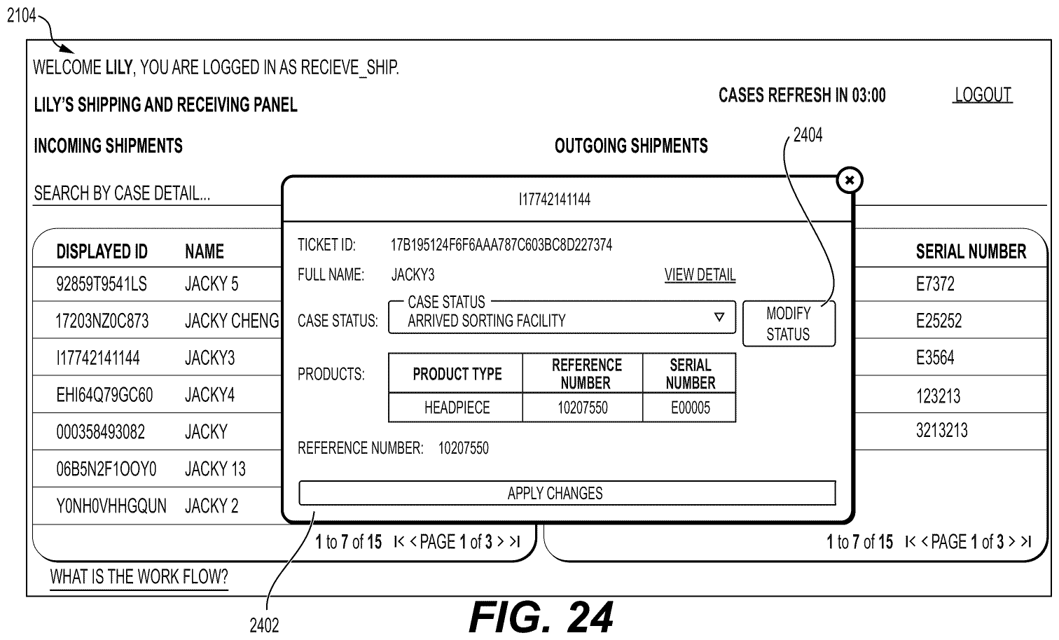

Any entry can be selected to produce a status screen 2402 for the selected repair order 1402 as depicted in FIG. 24. After the status is updated using the modify status button 2404, the status of the repair order is updated for both the repair supplier 110 and the end-user 102. The status change may also automatically trigger check boxes to be added next to one of more milestones 1502 associated with the repair order 1402.

If the status of any repair order 1402 is updated to “shipping back to end-user”, the shipping label generator and tracker 120 may automatically generate a shipping label and automatically add the tracking number to the repair order. Alternatively, the tracking number can be added manually. This would also trigger milestone 8 to be activated to indicate to the end-user 102 that the repair has been shipped and is on route.

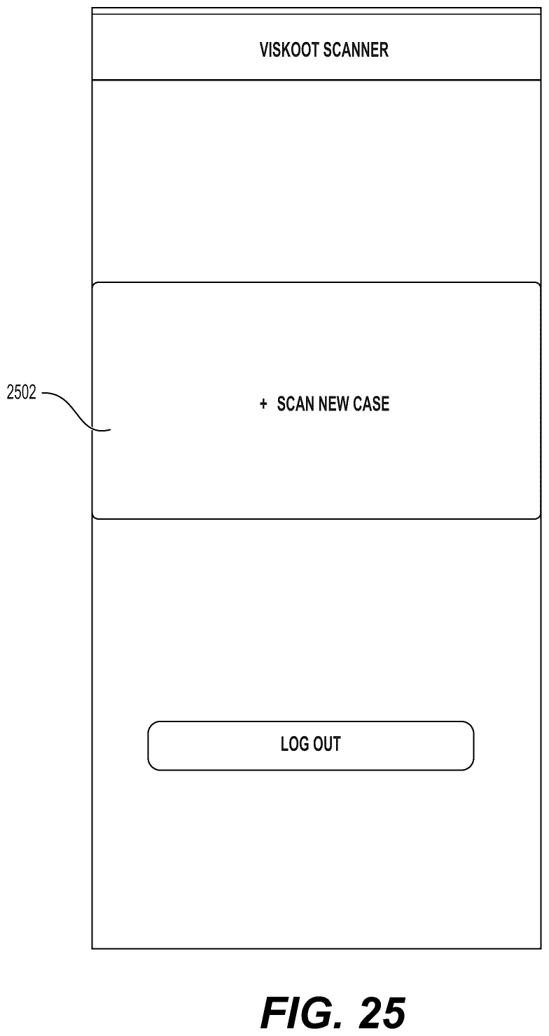

As products arrive at the repair supplier 110, the machine-readable code 1310 that was affixed to the packaging by the end-user 102 can be scanned by selecting the scan new case button 2502 from within mobile application 104. An example scan new case button 2502 is depicted in FIG. 25. The scanning of the machine-readable code 1310 can be accomplished using dedicated hardware, such as a 1D or 2D barcode scanner, or can be imaged using any device capable of analyzing and decoding machine-readable codes.

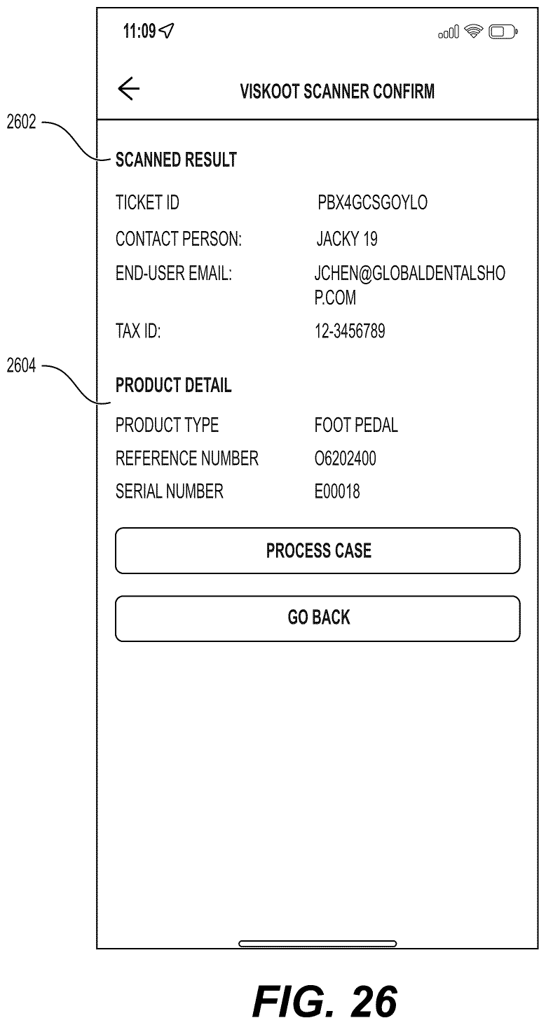

As previously mentioned, the machine-readable code 1310 may include one or more identifying pieces of information such as confirmation number 1302, shipper name, etc. The decoded information 2602 from the machine-readable code 1310 is displayed next to the product detail information 2604 as depicted in FIG. 26. The repair order 1402 may automatically be assigned to an available technician according to predefined rules or availability. Alternatively, the repair order 1402 can be manually assigned to a technician by a supervisor.

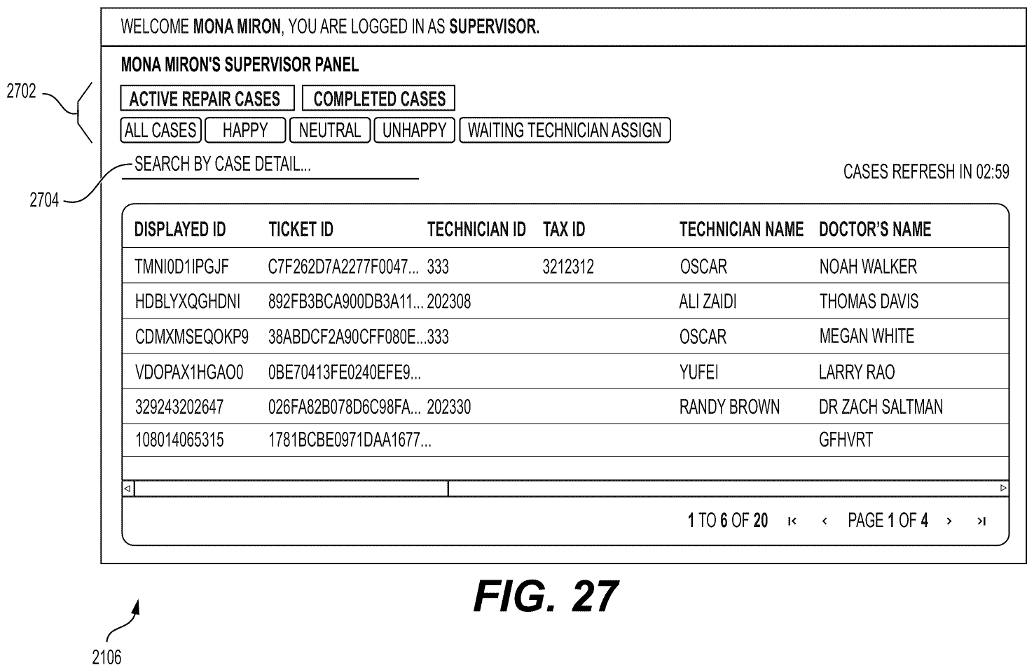

FIG. 27 depicts an example supervisor panel 2106. Supervisor panel 2106 provides supervisors with real-time insight of active repairs and completed cases. This allows for effective resource allocation and ensures a smooth workflow throughout the case management process. In addition, search function 2704 enhances efficiency by locating specific cases.

The supervisor panel 2106 includes a plurality of filters 2702 that can be used to limit the displayed results. For example, a “waiting technician assignment” filter 2702 can be used to see any remaining cases that were not assigned to a technician for one or more reasons.

To help supervisors quickly grasp the sentiment of each repair order 1402, different colors are used to indicate the urgency of each case based on end-users' feedback that they provide during their case submission. Supervisors can consider also including a special token of appreciation, such as a future credit or discount, to encourage certain end-users 102 to become repeat customers or brand ambassadors.

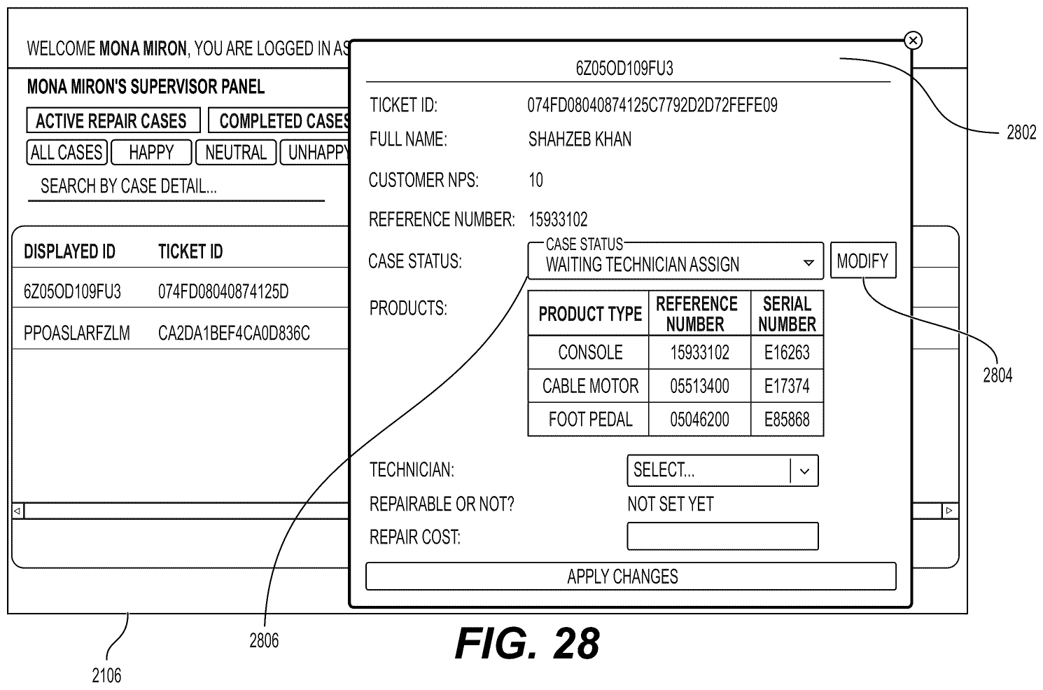

By clicking on any repair order 1402, additional information 2802 about the repair order 1402 is displayed to the supervisor as depicted in FIG. 28. The supervisor can change the case status 2806 to “inspecting” and choose a technician by selecting the modify selection 2804. Adding a repair order 1402 to a technician automatically adds the case to the technicians list of cases for repair.

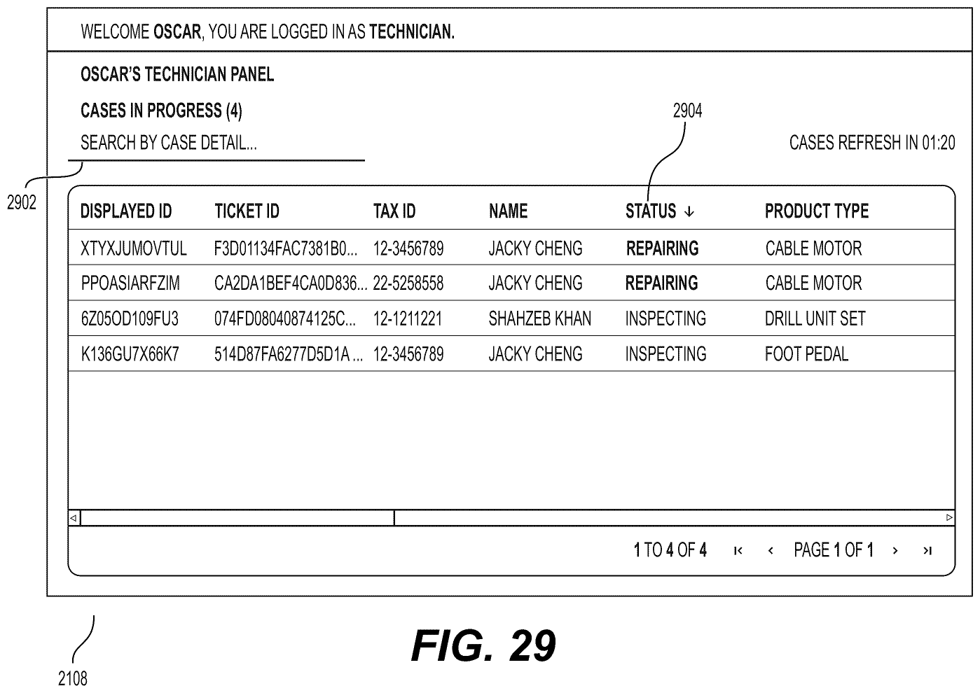

FIG. 29 depicts an example technician panel 2108. Each technician at the repair supplier 110 is provided with their own technician panel 2108 which only lists their currently active cases. For example, if a technician marks a repair order 1402 as “ready for shipping,” the repair order is removed from the technician panel 2108 to the receive shipment panel 2104.

Technician panel 2108 panel shows cases in progress and cases needing a response. The status of each case is indicated in status column 2904. It is designed to assist technicians in identifying different cases and their statuses. A search function 2902 is provided to allow the technician to find certain cases if the list is long.

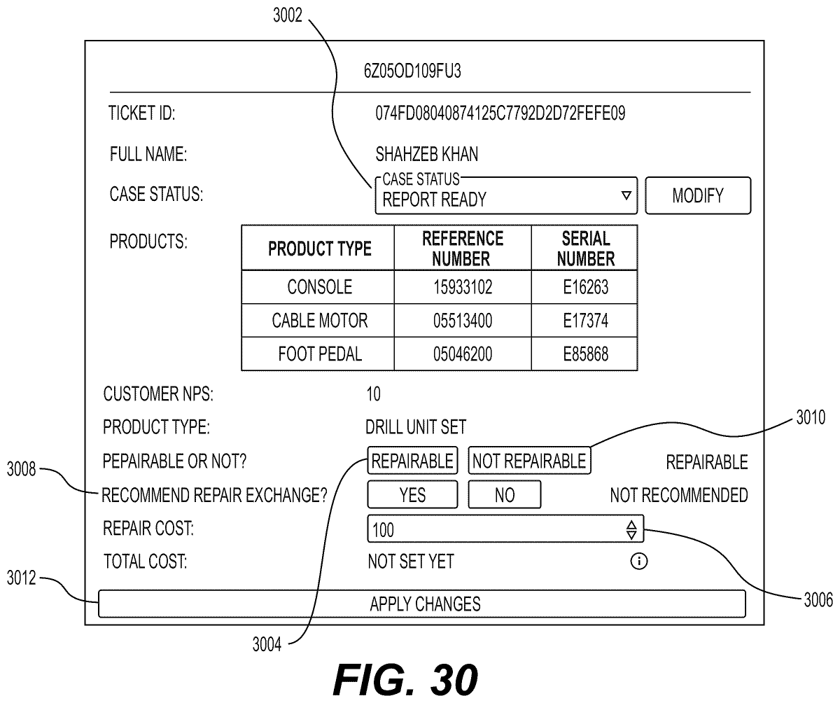

After cases are added to the technician panel 2108 by a supervisor or automatically, they are assigned an inspecting status. After inspecting the product and preparing an inspection report, the technician can select the case and update the case status 3002 to “report ready” as depicted in FIG. 30. If the product is repairable, the technician selects repairable option 3004 and fills out the repair cost 3006 based on the quotation. Then the technician can choose whether to recommend the repair exchange program 3008 to the end-user 102. If the product is not repairable, the technician selects not repairable option 3010. Selecting apply changes 3012 causes the status to be updated and triggers sixth milestone 1502 to have a check mark (FIG. 15). The information entered by the technician determines if the end-user receives the screen type depicted in FIG. 16, 17, or 18.

Once the end-user 102 makes a final decision to proceed with repairs and pays the repair fee, the case status 2904 changes to “repairing” on the technician panel 2902. The technician can then begin the repair process. After finishing the repair, the technician updates the case status 3002 to “Finish repairing” and clicks apply changes 3012. The case is marked as “waiting for ship out” and sent to the shipping department.

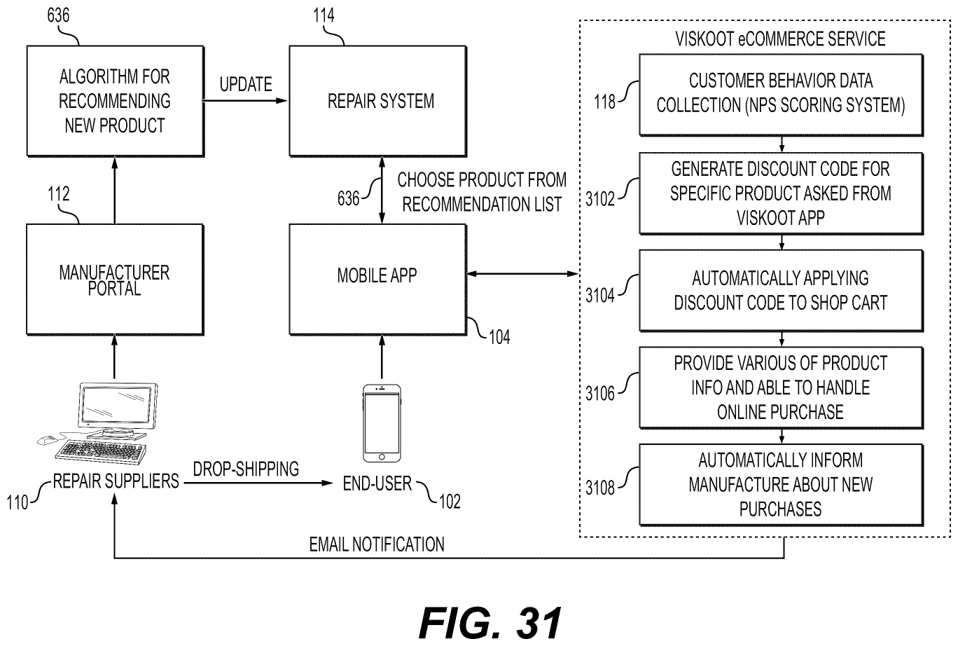

FIG. 31 depicts an embodiment of the product recommendation feature 636. As previously explained with respect to FIG. 19, the end-user 102 may be provided with a product recommendation if a repair is not possible. The product recommendation 636 may be based on customer behavior data collected by behavior data collection 118. From the customer behavior, a discount code may be generated in step 3102. The discount code amount may be based on the status of the end-user 102, purchase price, associations, etc. as is known in the art. The discount code is then automatically applied to the purchase price in step 3104 and as depicted in FIG. 19. The information provided to end-user 102 includes product information and any other necessary information to handle the purchase in step 3106. In some embodiment, the repair supplier or manufacturer 110 is automatically notified of any replacement purchases in step 3108.

The Product Recommendation feature is applied when the user wants to participate in the product exchange program. This means; If the user does not want to proceed with a repair or if the product cannot be repaired, they will have the option to participate in the exchange program. When the user clicks on the exchange program option, the prescriptive AI which is embedded within the Viskoot app will recommend a list of products available for exchange, recommendations are based on the original product. Viskoot can recommend the exact product or the newer model/generation. After they select a product, the app will generate a discount code for them on the Viskoot eCommerce service website. The user will be redirected to the eCommerce website's checkout cart with the discount applied to the selected product. Once the user completes the payment, the Viskoot app will inform the manufacturer about the new order (exchange program) details. The manufacturer will then ship the new product to the customer.

### Power Tool Repair

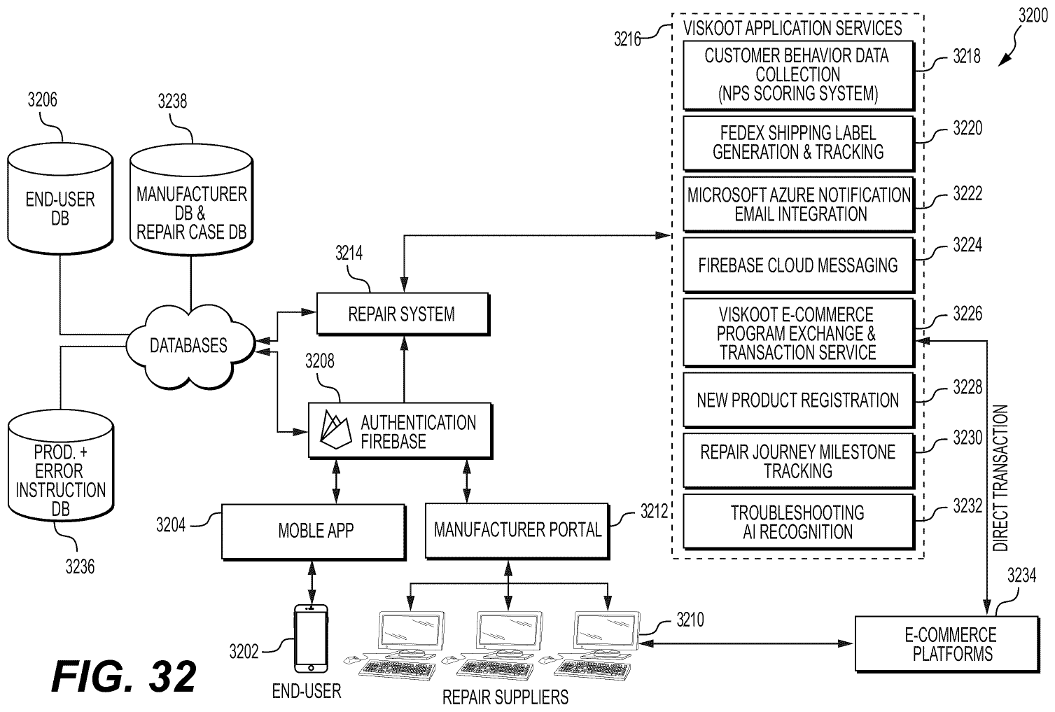

As previously discussed, repair management system 100 can also be configured to handle the repair and replacement of power tools for consumers. A repair tool management system 3200 (similar to repair management system 100) will now be described. Referring first to FIG. 32, depicted is a network diagram of repair management system 3200 showing the connection between end-users 3202, manufacturers, and repair technicians through the mobile application 3204 and back-end. End-users 3202 can access mobile application 3204 by downloading a dedicated mobile application to a smartphone or tablet. Alternatively, the end-user 3202 can access a web app through a browser that provides similar functionality to the mobile application 3204. To use mobile application 3204, each end-user 3202 must register with repair management system 3200. Identifying information such as name, company, address, etc. is collected from each end-user 3202 and is stored in the user database 3206. This allows all repairs received within repair management system 3200 to be associated with a particular end-user 3202.

In a preferred embodiment, Firebase Authentication Service 3208 is utilized because it supports authentication using passwords, phone numbers, and log-in through popular identity providers like Google, Facebook, Twitter, etc. Firebase Authentication Service 3208 can also support other forms of authentication such as fingerprint identification, multi-factor authentication, or facial recognition. It should be apparent to one of ordinary skill in the art that any secure authentication system may be utilized.

In some embodiments, firebase authentication service 3208 is utilized to authenticate both end-users 3202 and repair suppliers 3210. However, separate systems may be utilized if different levels of authentication are needed.

Repair suppliers 3210 may be third-party repair technicians or manufacturers. Access to repair management system 3200 for repair suppliers 3210 can be accessed through manufacturer portal 3212 using a dedicated mobile application (like with end-users 3202) or a web application provided through a browser.

Communication and interaction between mobile application 3204 and manufacturer portal 3212 is managed by repair system 3214. Further, repair system 3214 also controls the various application services used to manage repair management system 3200. Application services 3216 may include but are not limited to, customer data collection system 3218, shipping label generator & tracker 3220, notification system 3222, messaging system 3224, financial transaction system 3226, product registration system 3228, repair update and tracking system 3230, and AI module 3232.

In some embodiments, the mobile application 3204 may suggest new or replacement items to end-users 3202 if it is determined that repairs cannot be completed. For example, manufacturer database 3238 can store a list of related or suitable replacement items in association with each power tool. End-users 3202 can utilize e-Commerce platform 3234 to purchase any needed power tool directly from the manufacturer or be directed to a third-party website for the same item.

### Mobile Application 3204 and Interface

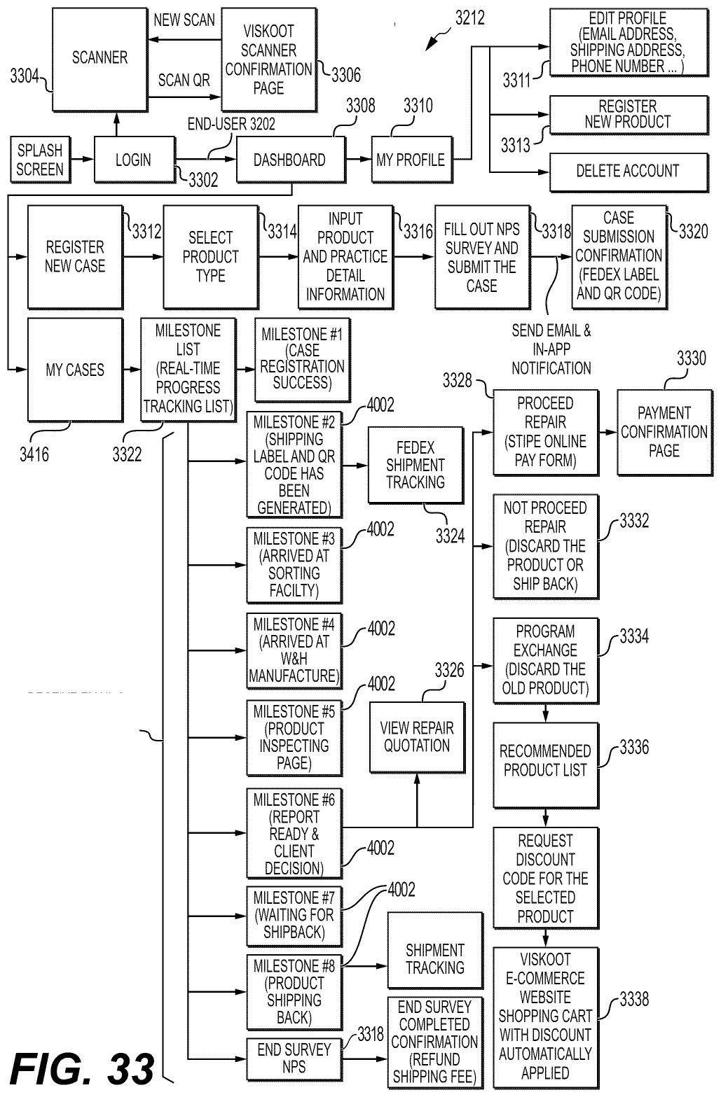

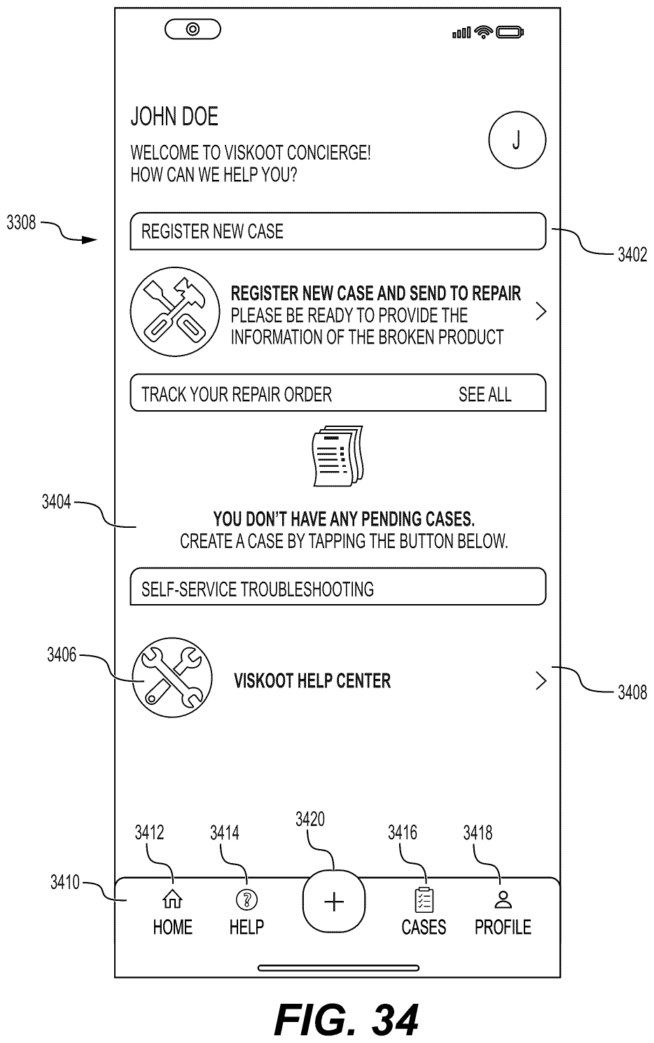

FIG. 33 depicts a flowchart showing the capabilities of mobile application 3204. The mobile application 3204 allows the end-user 3202 to login in step 3302 and be authenticated through Firebase authentication service 3208. After an end-user 3202 is authenticated in step 3302, a dashboard 3308 is displayed to the end-user 3202 as depicted in FIG. 34. In a preferred embodiment, the dashboard 3308 is displayed to every end-user 3202 after being authenticated. The dashboard 3308 includes sections for registering new products 3402 that can be used to register any Power tool in case later repair requests are needed. This allows end-users 3202 to provide the Power tool information one time and it is saved in association with the end-user information in user database 3206. The tracking section 3404 provides the end-user 3202 with a listing of all active repair requests. If additional help is needed with the mobile application 3204, the end-user 3202 can select 3406 to be directed to a help center 3408. The help center 3408 may include frequently asked questions (FAQ) to provide the most common answers or additional options like the ability to chat or call a live agent to discuss the issue.

In some embodiments, the bottom of dashboard 33080 includes access menu 3410 which is static and is displayed on most pages of mobile application 3204. Access menu 3410 may provide a home option 3412 that can be used to return to dashboard 3308 at any point, a help option 3414 to access additional help related to the mobile application 3204, a cases option 3416 allowing the end-user 3202 to view all active and closed repair requests, and a profile option 3418. If the end-user 3202 selects the profile option 3418, a my profile screen 3310 is displayed listing the profile information saved in mobile application 3204. The end-user 3202 can select any of the displayed fields such as e-mail address, address, shipping address, phone number, etc. in step 3311 to provide new or updated profile information. After any changes are made to the information on my profile screen 3310, the user database 3206 is updated to reflect the changes. The end-user 3202 can also register new power tools in association with their account using register new produce screen 3313. The dashboard 3308 also comprises a plus option 3420. The plus option 3420 can be used to initiate a repair request without the end-user having to return to the dashboard 3308.

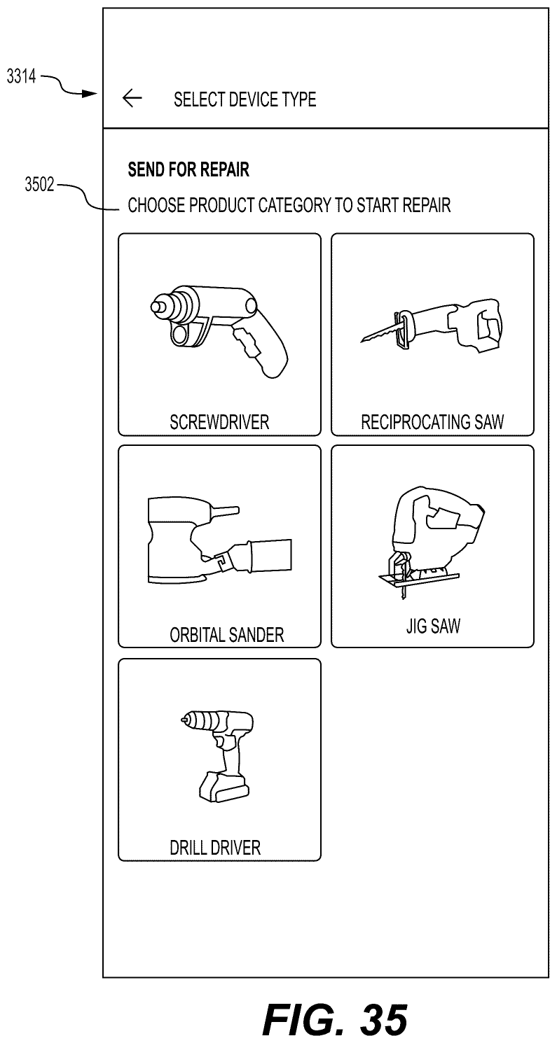

If the end-user 3202 selects the plus option 3420 or register new case option 3402, a new repair request is initiated in step 3312 so that all necessary information regarding the repair request can be collected. In a preferred embodiment, a select product type screen 3314 is first displayed to the end-user 3202 so that a category 3502 can be selected as depicted in FIG. 35.

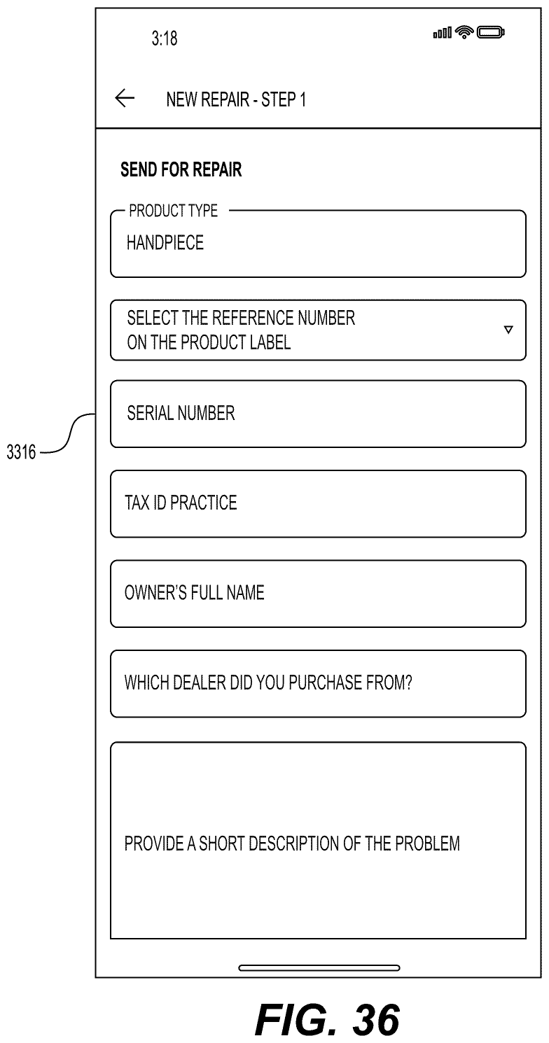

The end-user 3202 is then prompted to enter additional product information on product information screen 3316. An example product information screen 3316 is depicted in FIG. 36. The additional product information may be the product's reference number, serial number, tax ID, the name of the owner or operator of the product as well as a short description of the repair issue.

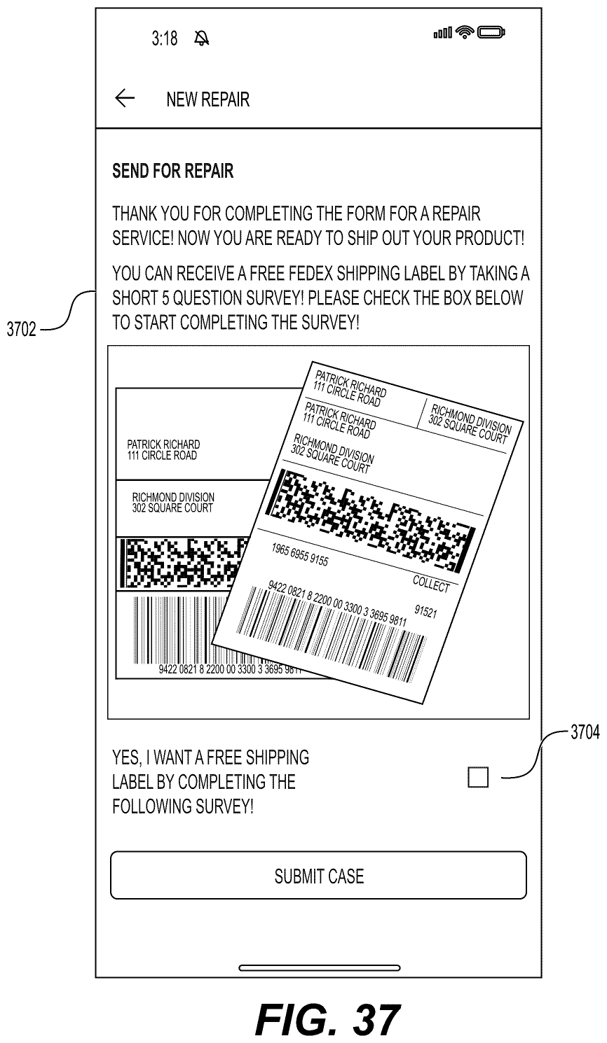

If the power tool is a small product that can be shipped, the end-user 3202 may be provided with a shipping option screen 3702 as depicted in FIG. 37 informing them that they can receive free shipping by filling out a small survey. If the end-user 3202 selects check box 3704, the mobile application 3204 displays survey screen.

In a preferred embodiment, each product stored in error and product database 3236 is associated with a particular repair location for that item. The repair location may be a third-party repair service 3210 or the manufacturer's own repair service. The manufacturer can select their preferred repair location for each of their products. For example, a manufacturer may choose to have products of a first type be repaired by a particular third-party repair shop whereas products of a second type are shipped directly to the manufacturer. Because the repair location information is stored ahead for each power tool device, the repair location address can automatically be pulled by repair management system 3200. The end-user 3202 does not have to determine the address. The end-user 3202 is only required to affix a generated shipping label to the power tool device packaging as it already has the correct address specified in advance by the manufacturer.

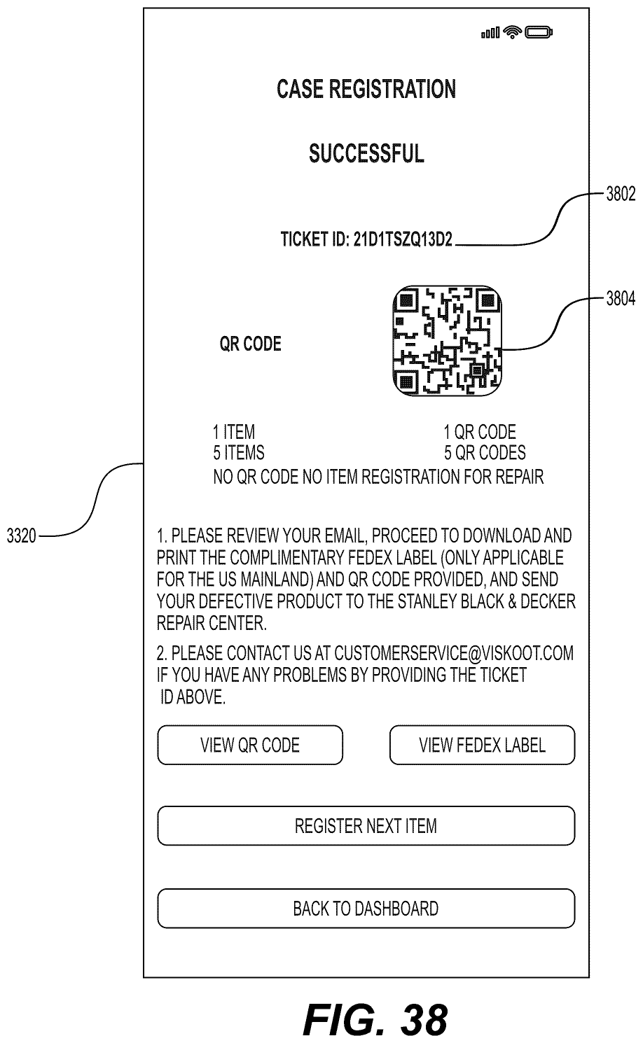

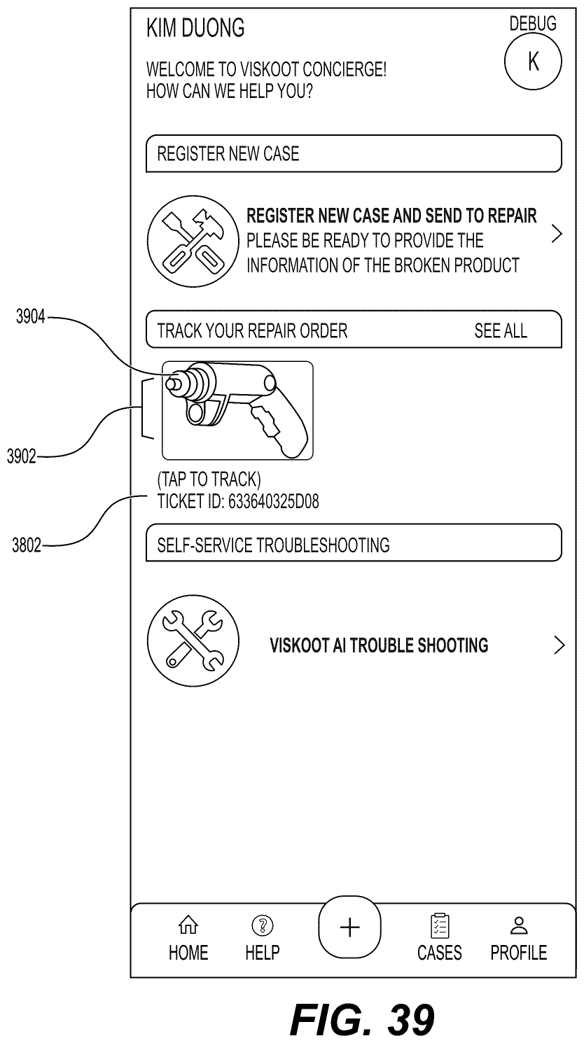

After the end-user 3202 completes the (optional) survey 3318 if check box 3704 was checked, a submission screen 3320 is displayed to the end-user 3202. An example submission screen 3320 is depicted in FIG. 38. The end-user 3202 is provided with a Confirmation Number 3802 which is used to uniquely identify each repair request. The Confirmation Number 3802 may comprise a combination of alphanumeric characters and numbers as is known in the art. The Confirmation Number 3802 is stored in association with all of the repair request information gathered. The tracking number for the shipping label is also stored in association with the Confirmation Number 3802.

In a preferred embodiment, the machine-readable code 3804 that is generated encodes information related to the repair such as Confirmation Number 3802, the tracking number, the address, etc. The amount of information that can be included in the machine-readable code is only limited by the capabilities of the type of machine-readable code. For example, a 1D barcode can generally only include up to 85 encoded characters whereas a 2D barcode, such as a QR code, can encode 3000+ characters if needed. The encoded information in the QR code may optionally be encrypted if the characters include sensitive personal information such as the shipper's return address.

The repair order 3902 is then added to dashboard 3308 as depicted in FIG. 8. The repair order 3902 preferably includes an image 3904 of the Power tool being repaired and/or the Confirmation Number 3802. The end-user 3202 can select repair order image 3904 to view case milestones associated with repair order 3902.

FIG. 9 depicts an example milestone screen 3322 showing the real-time status of the repair process for repair order 3902. For example, after the end-user 3202 has shipped the Power tool, the second milestone 4002 can be selected by the end-user 3202 to view the shipping status. The tracking information associated with the Confirmation Number 3802 can be used to retrieve the associated tracking number and the end-user 3202 can be directed to a screen or website 3324 showing the current shipment status (e.g., in transit, arrived, etc.). As each milestone 4002 in the repair process is completed, an icon 4004 associated with the milestone 902 is changed to reflect completion. For example, check marks can be added to the icons 4004 for each milestone 4002 which allows the end-user 3202 to quickly identify the current status through a visual inspection.

As will be discussed later, the additional milestones 4002 receive check marks as the Power tool proceeds through repair. The Confirmation Number 3802 is also used by the repair facility to track the repair process. As each repair step is completed and the information is updated, the corresponding milestone 4002 is activated. For example, when a shipment arrives at the repair facility, the QR code is scanned and the status of the case 3902 is changed to “received.” This triggers the third milestone 4002 to be changed to a check mark. Various actions, or combinations of actions, performed at the repair facility can be used to activate or deactivate certain milestones 4002. The activation of each milestone 4002 may also cause certain other actions to be performed such as sending a text message, email, etc. The rules for triggering each milestone 4002 are controlled by repair update and tracking system 3230 and are configurable by a technician or manufacturer.

No action is required by the end-user 3202 until the sixth milestone 4002 is activated which indicates that the repair report is ready. Generally, when the repair report is ready, the end-user 3202 will be provided with one or more notifications because the end-user 3202 must select a repair option.

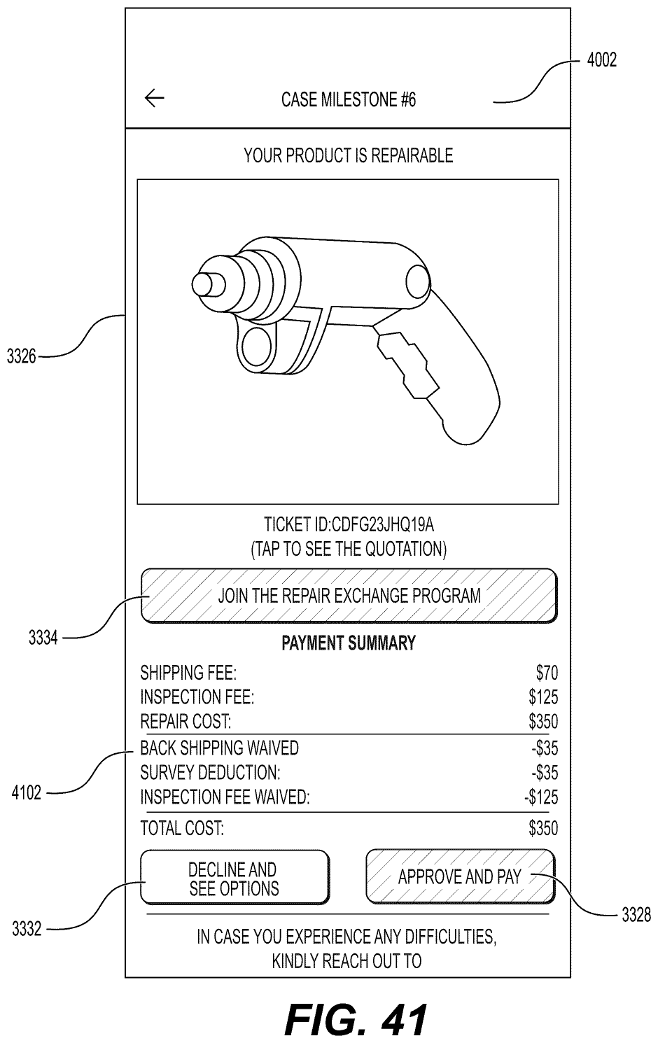

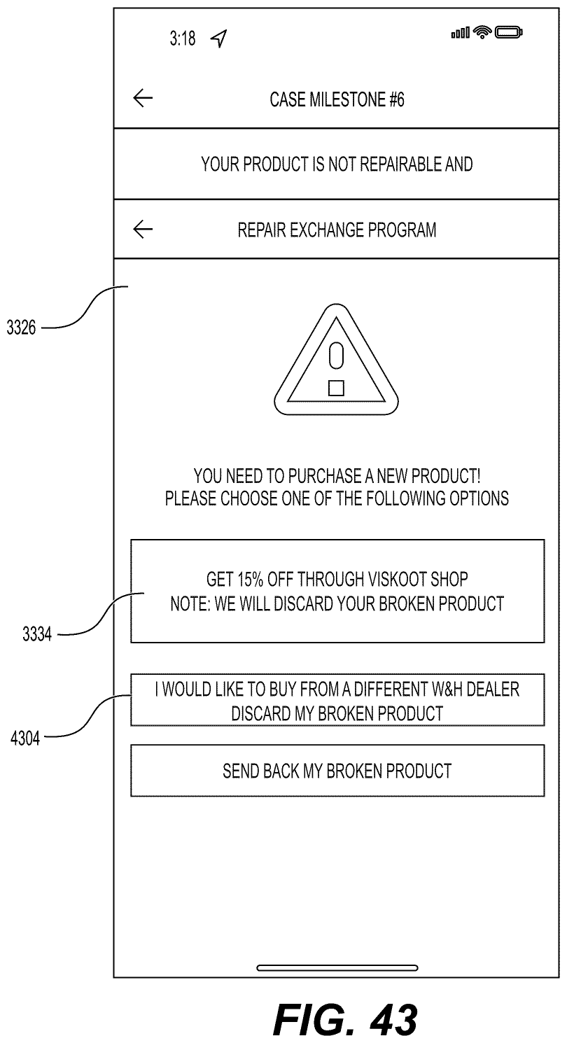

Activation of the sixth milestone 4002 causes a repair report screen to be displayed. Example repair report screens 3326 are depicted in FIGS. 41-43. The content of the repair report determines which repair report screen 3326 is displayed to the end-user 3202. If the Power tool can be repaired, the repair report screen 3326 displayed in FIG. 41 is displayed. The end-user 3202 is informed of the repair cost in payment summary section 4102 which includes itemized costs and reflects and discounts for the end-user 3202. If the end-user 3202 selects the approve option 3328, the end-user 3202 can utilize financial transaction system 3226 to pay for the repairs. A payment confirmation page 3330 is generated informing the end-user 3202 that payment is completed and the requested repair will be completed. A payment confirmation is generated after the repair has been approved and paid for. If the repair is no longer required, the end-user 3202 can select decline option 3332 and choose to have the Power tool returned or destroyed.

The end-user 3202 can also choose product exchange option 3334 which allows the end-user 3202 to purchase a similar or replacement Power tool at a discount. This option can be used to direct end-users 3202 to purchase replacements directly from the manufacturer instead of a third party without the end-user having to exit the mobile application 3204.

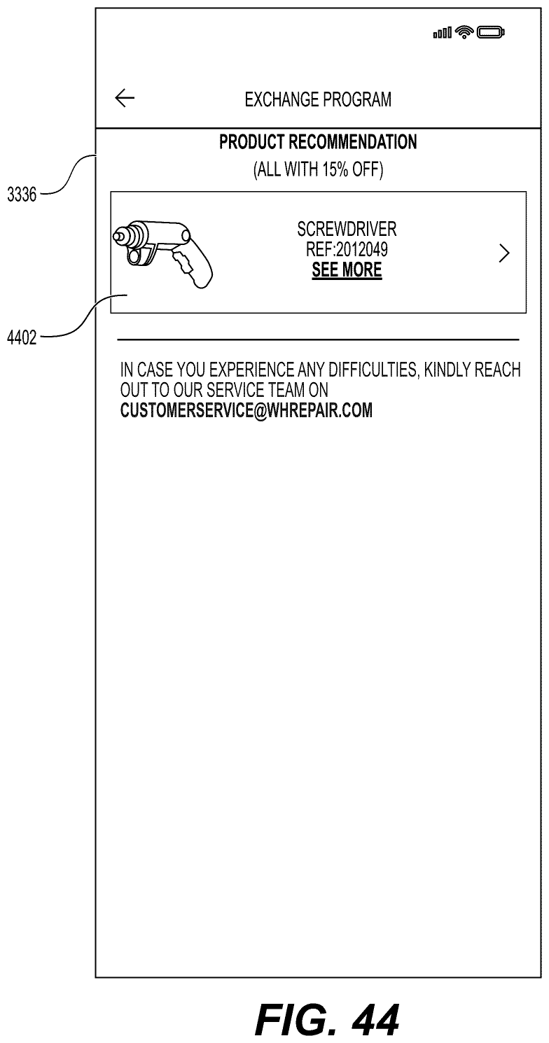

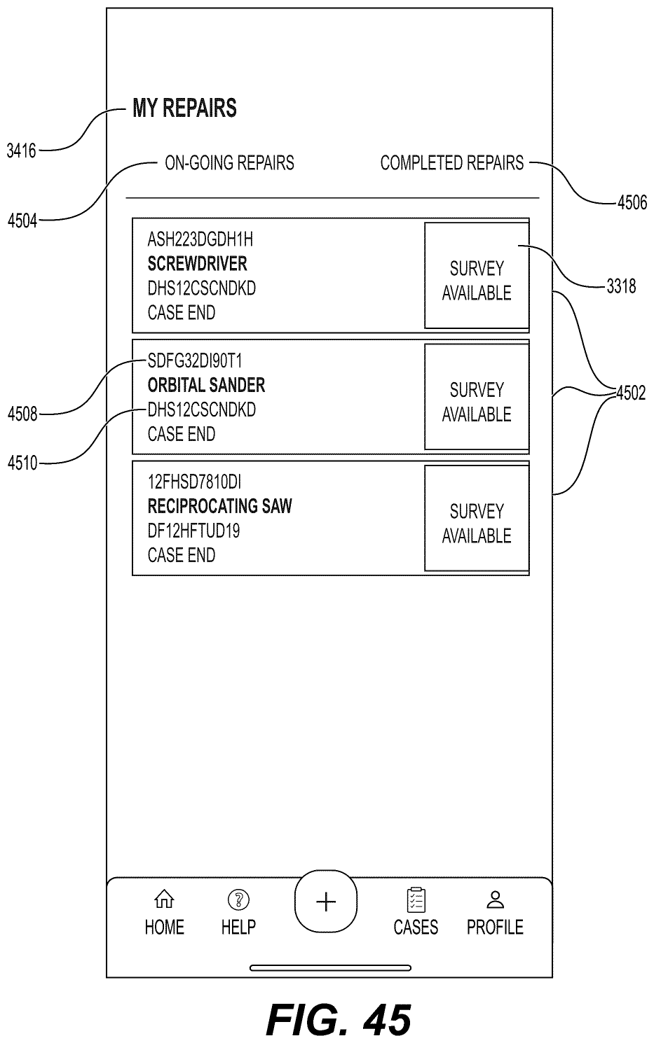

Selecting product exchange option 3334 directs the end-user 3202 to a product recommendation page 3336 listing products that are suitable as a replacement. The end-user 3202 can select one of the displayed recommendations 4402 and pay for the purchase using the financial transaction system 3226 as depicted in FIG. 44.

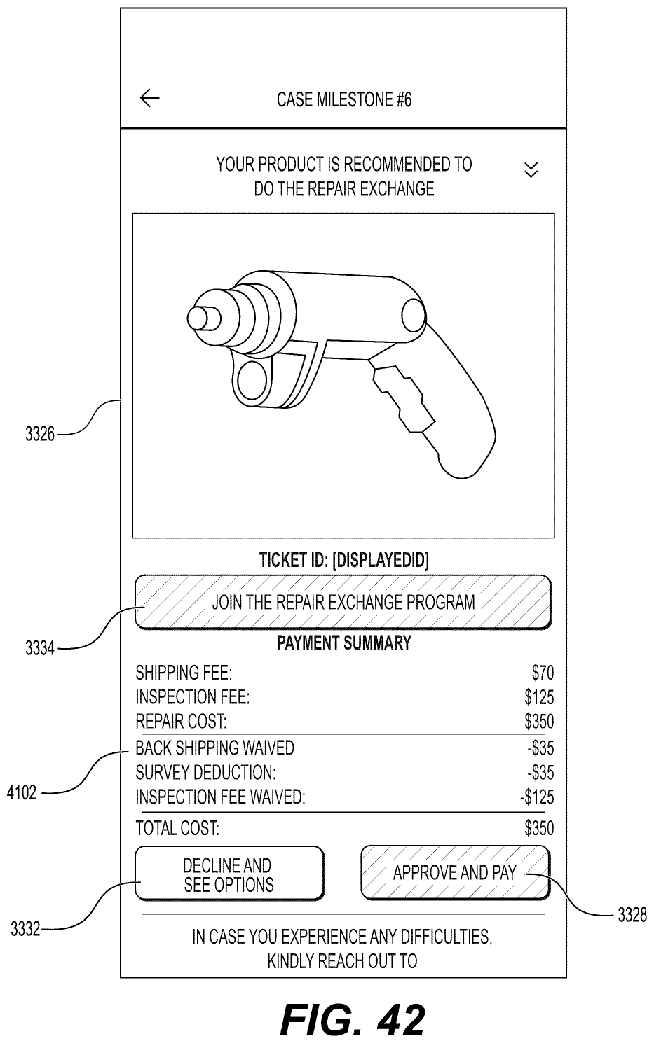

In certain instances, the repair cost may be comparable to or greater than the cost of a completely new or refurbished product. If a replacement cost is within a certain predefined threshold of the repair cost or greater than the repair cost, the repair report screen 3326 of FIG. 42 may be displayed to the end-user 3202 indicating that the repair exchange program 3334 can be joined. If the end-user 3202 would like to decline the repair, they can select decline option 3332. However, the end-user 3202 is still free to also choose to have the repair performed by selecting approve option 3328.

The repair report screen 3326 may also indicate that the product is not repairable. The end-user 3202 can select product exchange option 3334 to buy a new product at a discount or select decline option 3332 and choose to have the product returned or destroyed. If the end-user chooses to have the object returned, the end-user is prompted to pay for the return shipping by financial transaction system 3226.

Returning to FIG. 34, if end-user 3202 chooses cases option 3416 from dashboard 3308, a listing 4502 of all repairs associated with the logged-in end-user 3202 are displayed. The end-user 3202 can toggle the display by selecting the ongoing repair option 4504 or the completed repair option 4506 to active or closed repair requests, respectively. For each repair request, identifying information 4508 is provided adjacent to the most recent status 4510. In some embodiments, the most recent status 4510 may indicate that a survey 3318 is available to the end-user 3202 related to the repair. The end-user 3202 can fill out the survey 3318 to indicate their experience and satisfaction with the repair. Submitting the survey 3318 may result in a full refund for the return shipping cost of the repaired product.

### Manufacturer Portal 3212

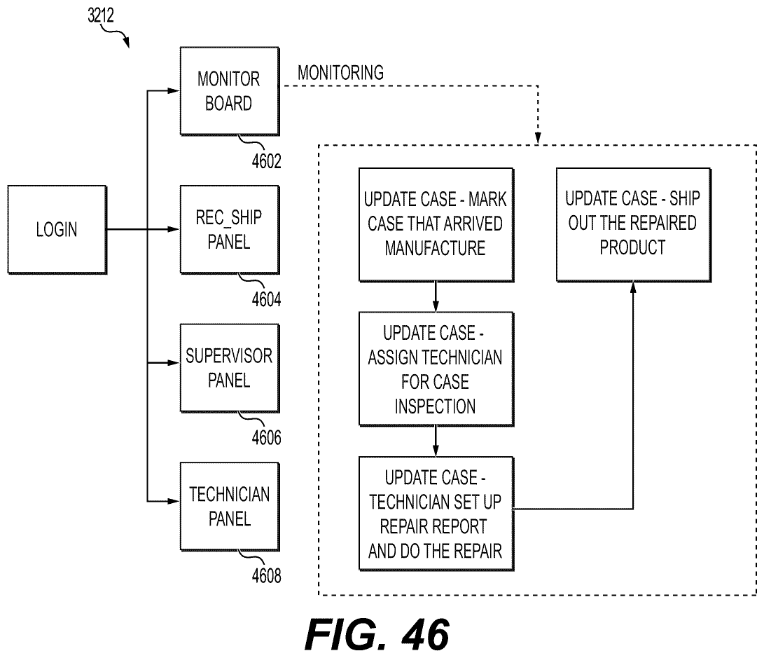

As previously discussed with reference to FIG. 32, each repair supplier 3210 has access to manufacturer portal 3212 which can be used to scan received repairs, track the status of repairs, and ship out completed repairs. The main elements of Manufacturer Portal 3212 are depicted in FIG. 46. Manufacturer Portal 3212 generally comprises monitor board 4602, receive shipment panel 4604, supervisor panel 4606, and technician panel 4608. Each user of Manufacturer Portal 3212 can be assigned one or more designations or access levels which determine which of the features of Manufacturer Portal 3212 can be accessed. For example, upper management may only be provided access to monitor board 4602 whereas a supervisor may be provided access to both monitor board 4602 and supervisor panel 4606 but restricted from accessing technician panel 4608.

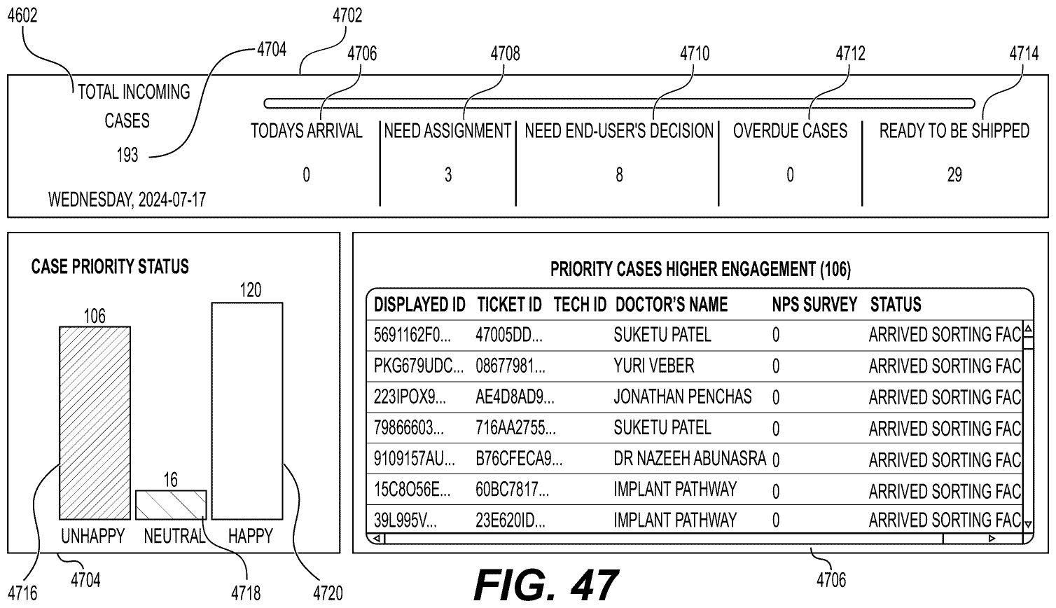

An example monitor board 4602 comprising three sections is depicted in FIG. 47. Monitor board 4602 generally comprises three parts: case number index 4702, case priority status 4704, and priority cases 4706. Case number index 4702 displays statuses related to daily or weekly statistics and may include the following categories:
Total Incoming Cases 4704 indicates the number of repair cases currently being sent to the shipping team.
Today's Arrival 4706 shows the cases that have arrived at the repair supplier 3210.
Need Assignments 4708 are the cases that have not been assigned to a technician yet and are now waiting to be assigned by the supervisor.
Waiting for End-user Decision 4710 includes cases where the technician has sent the repair report 3326 to the end-user 3202 and is awaiting their decision.
Overdue Cases 4712 indicates Cases that have exceeded the estimated repair or

inspection time of 24 hours.
Ready to be Shipped 4714 displays cases that have been repaired or returned and are ready to be shipped out by the shipment team.

To help the upper management and the whole team quickly grasp the status of each case, different colors may be used indicate the urgency of each case based on end-users' 3202 feedback that they provide during their case submission. Red may be used to represent unhappy end-users 3202 who are disengaged with the Power tool brand. These end-users 3202 can be highly prioritized and addressed immediately with a fast turnaround time. Green may be used to represent strongly satisfied end-users 3202 who are fully engaged with the Power tool brand. They can have repairs done within the normal turnaround time. Yellow may be used to represent neutral end-users 3202 who indicated neither strongly satisfied nor strongly dissatisfied feedback. They typically receive medium priority for a slightly higher than normal turnaround.

The case priority index 4704 shows the number of repairs falling into each of the different categories such as unhappy 4716, neutral 4718, or happy 4720. It should be obvious that the number of end-user categories and/or the display method used to represent each category can be varied as needed by each repair supplier 3210.

Priority Cases 4706 provides a listing of all cases marked red from unhappy end-users 3202. These cases require immediate attention as end-users 3202 are unhappy or have urgent repair needs. Priority cases 4706 may display information including Case ID, assigned technician, doctor's name, NPS survey result, and the current status. By referring to this table, upper management can easily identify these priority cases.

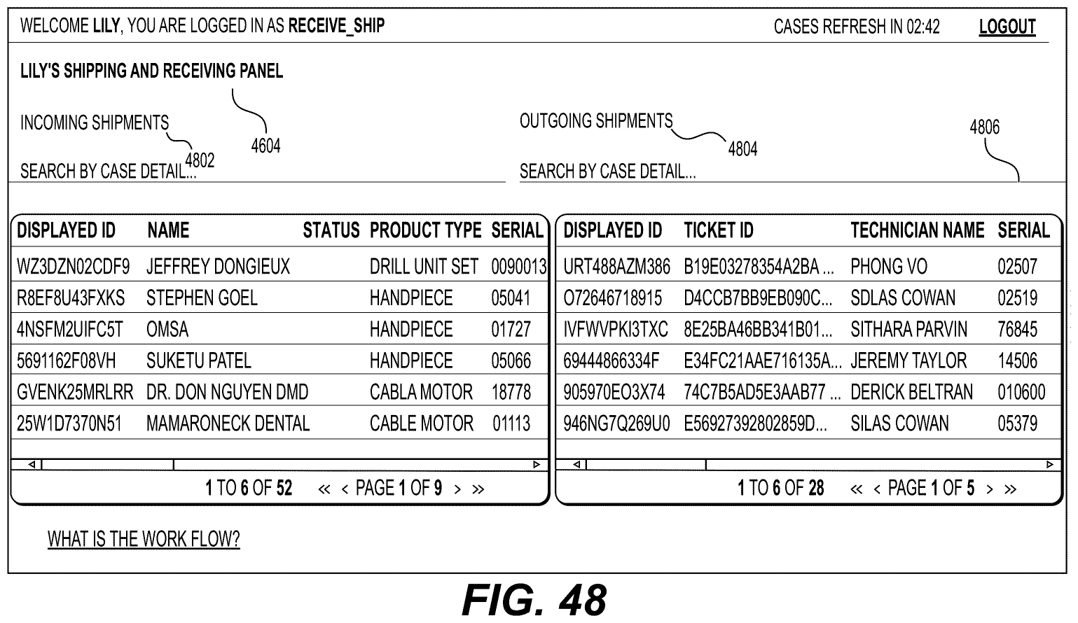

FIG. 48 depicts a sample receive shipment panel 4604. In a preferred embodiment, each member designated as mail room or shipping has access to receive shipment panel 4604 after being authenticated. The receive shipment panel 4604 is divided into two main sections, specifically designed to provide an overview of both incoming and outgoing repair cases. It also offers powerful search and modification features.

Incoming shipments section 4802 provides an organized list of all incoming cases destined for the repair supplier 3210 and displays corresponding statuses. Like in the monitor board 4602, the entries may be color-coded according to urgency based on end-users' 3202 feedback provided during their case submission. This clear view of the case statuses allows supervisors and technicians to prioritize and allocate resources effectively, ensuring prompt and efficient resolution.

The outgoing shipment section 4804 is similar to the incoming shipment section 4802 but provides information on cases that have been shipped from the repair supplier 3210. The incoming shipment section 4802 and outgoing shipment section 4804 also comprise search field 4806 used to locate specific cases by typing any identifying information such as Confirmation Number 3802, end-user name, etc.

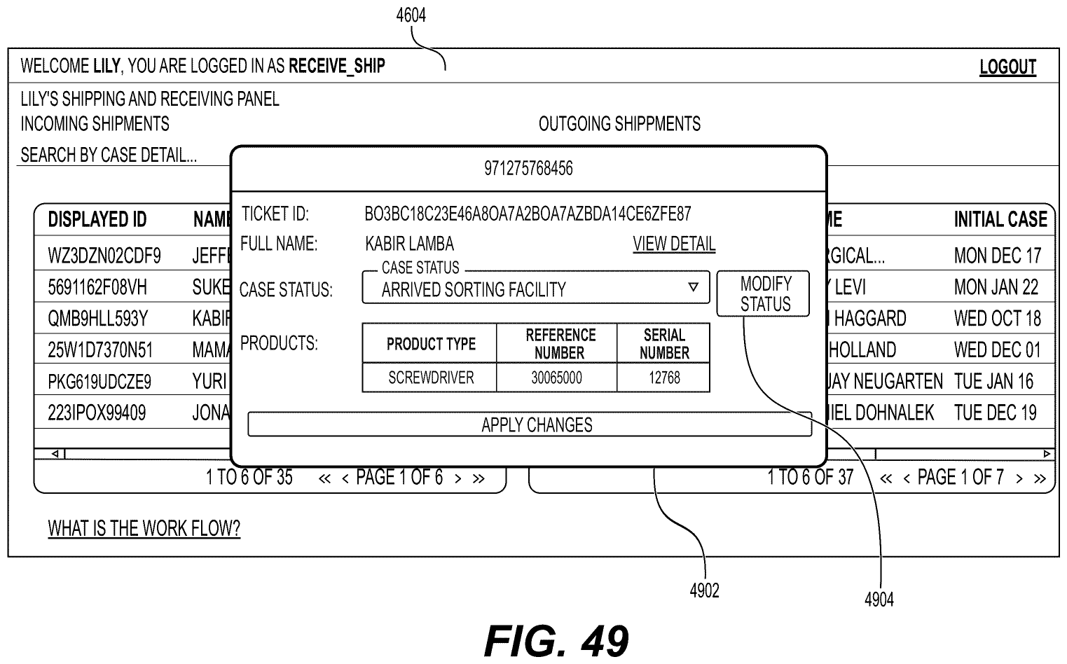

Any entry can be selected to produce a status screen 4902 for the selected repair order 3802 as depicted in FIG. 49. After the status is updated using the modify status button 4904, the status of the repair order is updated for both the repair supplier 3210 and the end-user 3202. The status change may also automatically trigger checkboxes to be added next to one or more milestones 4004 associated with the repair order 3802.

If the status of any repair order 3802 is updated to “shipping back to end-user” the shipping label generator and tracker 3220 may automatically generate a shipping label and add the tracking number to the repair order. Alternatively, the tracking number can be added manually. This would also trigger a milestone to be activated to indicate to the end-user 3202 that the repair has been shipped and is on route.

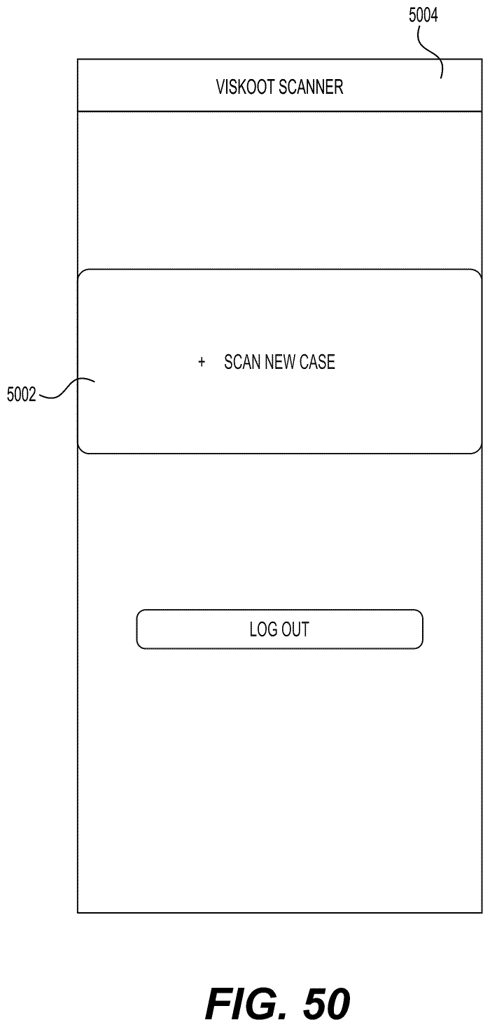

As products arrive at the repair supplier 3210, the machine-readable code 3804 that was affixed to the packaging by the end-user 3202 can be scanned by selecting the scan new case button 5002 from scanner page 5004 within mobile application 3204. An example scan new case button 5002 is depicted in FIG. 50. The scanning of the machine-readable code 3804 can be accomplished using dedicated hardware, such as a 1D or 2D barcode scanner, or can be imaged using any device capable of analyzing and decoding machine-readable codes.

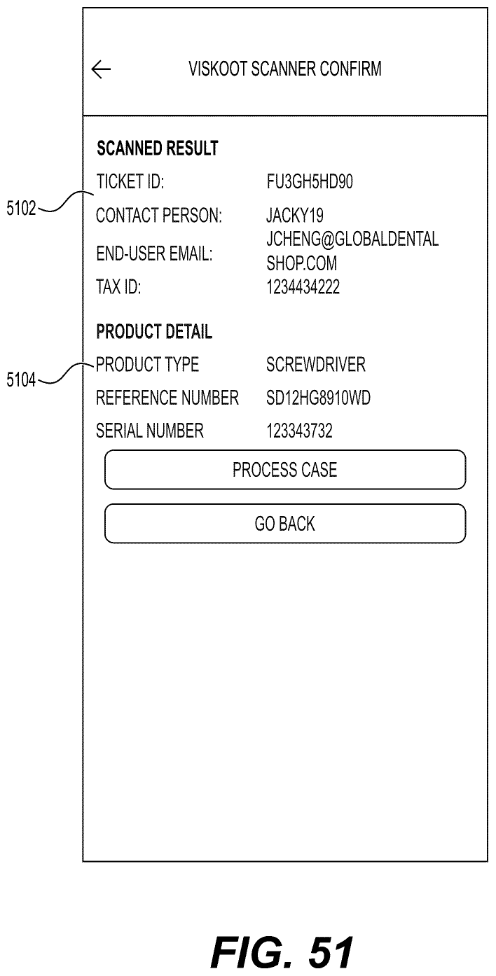

As previously mentioned, the machine-readable code 3804 may include one or more identifying pieces of information such as Confirmation Number 3802, shipper name, etc. The decoded information 5102 from the machine-readable code 3804 is displayed next to the product detail information 5104 as depicted in FIG. 51. The repair order 3802 may automatically be assigned to an available technician according to predefined rules or availability. Alternatively, the repair order 3802 can be manually assigned to a technician by a supervisor.

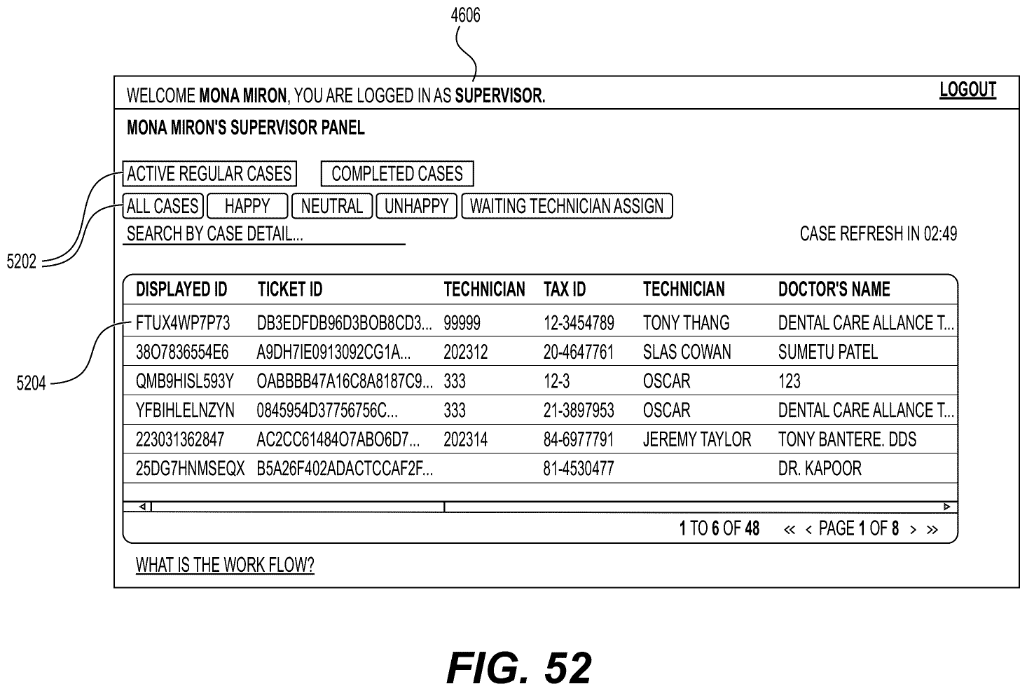

FIG. 52 depicts an example supervisor panel 4606. Supervisor panel 4606 provides supervisors with real-time insight of active repairs and completed cases. This allows for effective resource allocation and ensures a smooth workflow throughout the case management process. In addition, our search function will enhance your efficiency by locating specific cases. Simply type in the ticket ID or end-users name, and the corresponding case will appear.

The supervisor panel includes a plurality of filters 5202 that can be used to limit the displayed results. For example, a “waiting technician assignment” filter can be used to see any remaining cases that were not assigned to a technician for one or more reasons.

To help supervisors quickly grasp the sentiment of each repair order 3802, different colors are used to indicate the urgency of each case based on end-users' feedback that they provide during their case submission. Supervisors can consider also including a special token of appreciation, such as a future credit or discount, to encourage certain end-users 3202 to become repeat customers or brand ambassadors.

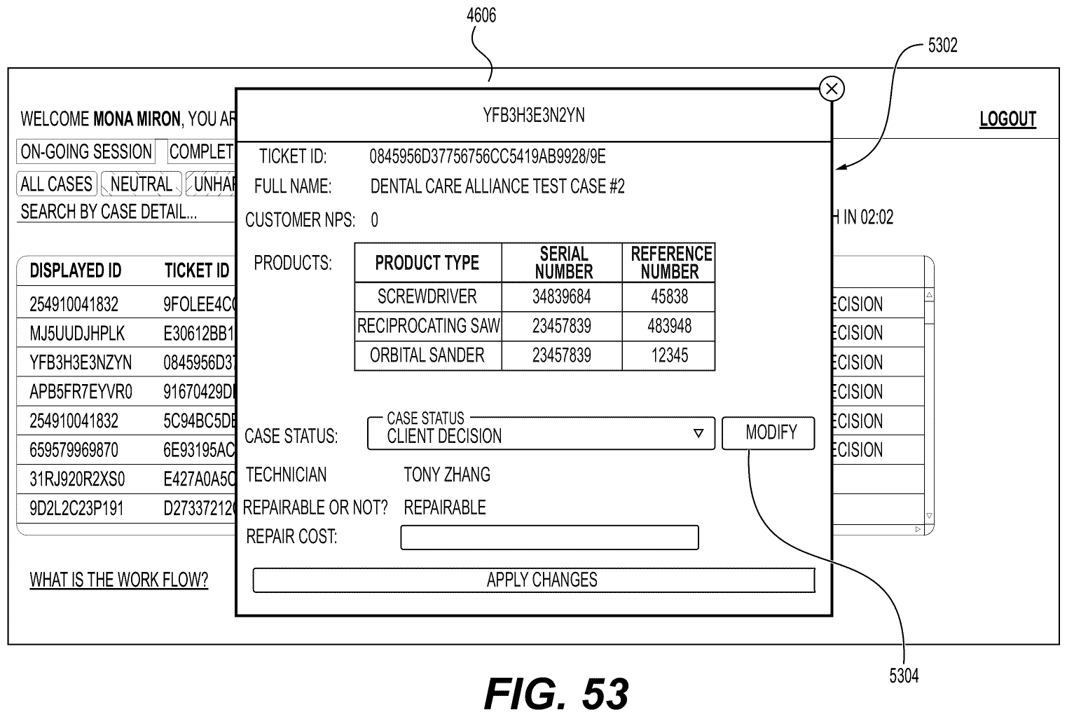

By clicking on any repair order 5204, additional information 5302 about the selected repair order 5204 is displayed to the supervisor as depicted in FIG. 53. The supervisor can change the case status to “inspecting” and choose a technician by selecting the modification selection 5304. Adding a repair order 5204 to a technician automatically adds the case to the technicians list of cases for repair.

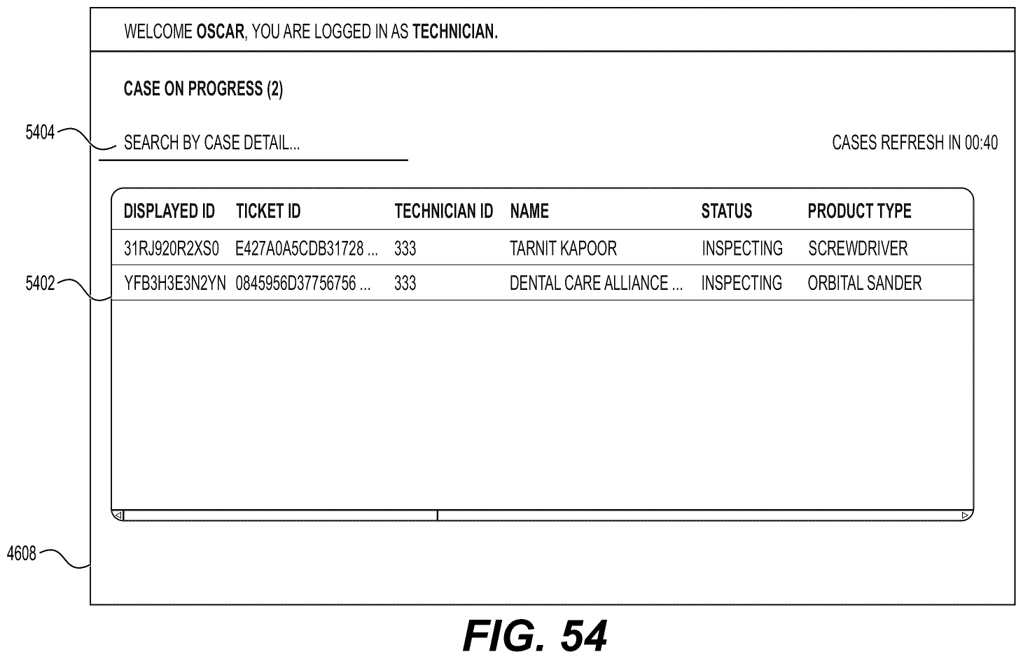

FIG. 54 depicts an example technician panel 4608. Each technician at the repair supplier 3210 is provided with their own technician panel 4608 which only lists their currently active cases. For example, if a technician marks a repair order 3802 as “ready for shipping,” the repair order is removed from the listing 5402 on technician panel 4608 to the receive shipment panel 4604.

Technician panel 4608 panel shows cases in progress and cases needing a response. It is designed to assist technicians in identifying different cases and their statuses. A search function 5404 is provided to allow the technician to find certain cases if the list is long.

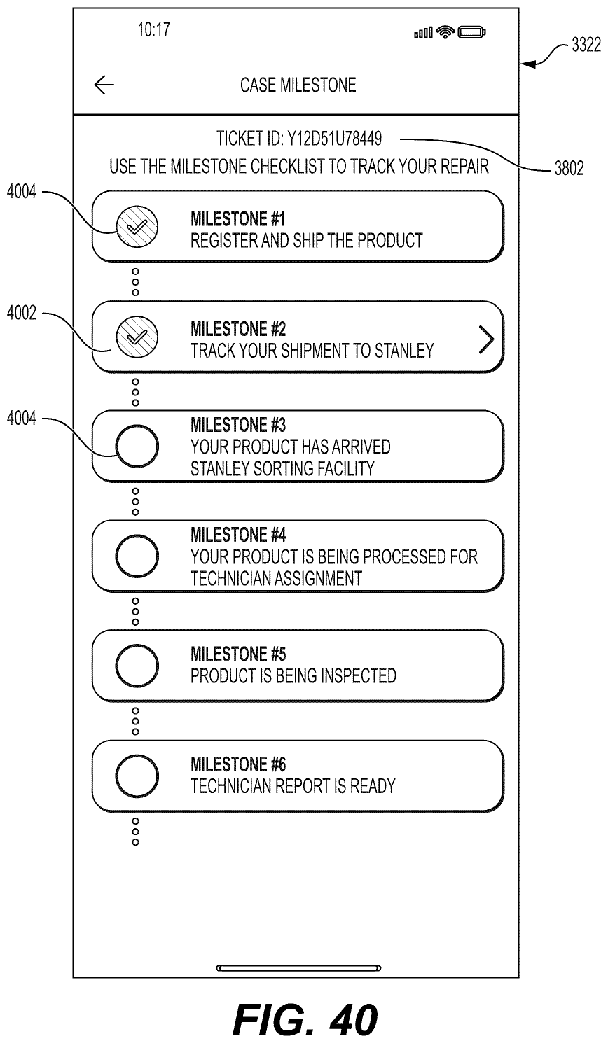

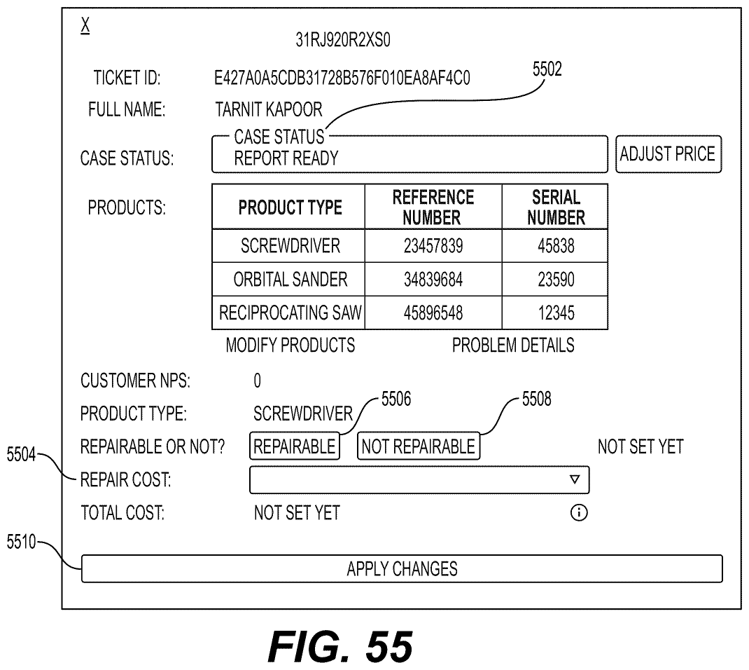

After cases are added to the technician panel 4608 by a supervisor or automatically, they are assigned an inspecting status. After inspecting the product and preparing an inspection report, the technician can select the case and update the case status 5502 to “report ready” as depicted in FIG. 55. If the product is repairable (5506), the technician needs to fill out the repair cost 5504 based on the quotation. Then the technician can choose whether to recommend the repair exchange program to the end-user. If the product is not repairable (5508), the technician should select “not-repairable.” Selecting apply changes 5510 causes the status to be updated and triggers sixth milestone 4002 to have a check mark (FIG. 40). The information entered by the technician determines if the end-user 3202 receives the screen type depicted in FIG. 41, 42, or 4312.

Once the end-user 3202 makes a final decision to proceed with repairs and pays the repair fee, the case status changes to “repairing” on the technician panel 2302. The technician can then begin the repair process. After finishing the repair, the technician updates the case status to “Finish repairing” and clicks “apply changes.” The case is marked as “waiting for ship out” and sent to the shipping department.

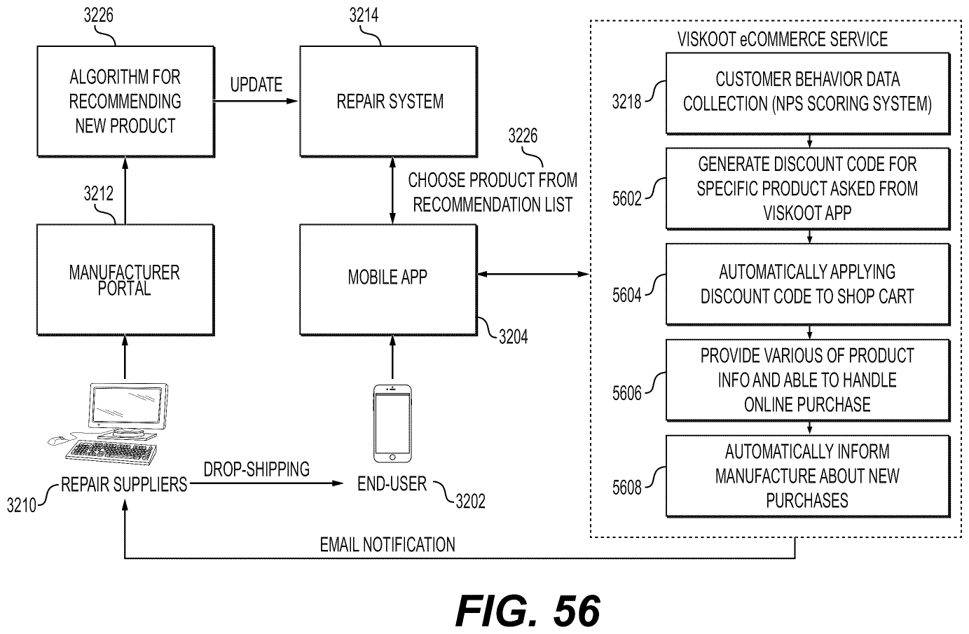

FIG. 56 depicts an embodiment of the product recommendation feature 3226. As previously explained with respect to FIG. 44, the end-user 3202 may be provided with a product recommendation if a repair is not possible. The product recommendation 3226 may be based on customer behavior data collected by behavior data collection 3218. From the customer behavior, a discount code may be generated in step 5602. The discount code amount may be based on the status of the end-user 3202, purchase price, associations, etc. as is known in the art. The discount code is then automatically applied to the purchase price in step 5604 and as depicted in FIG. 44. The information provided to end-user 3202 includes product information and any other necessary information to handle the purchase in step 5606. In some embodiment, the repair supplier or manufacturer 110 is automatically notified of any replacement purchases in step 5608.

While specific embodiments of the invention have been described above, it will be appreciated that the invention may be practiced other than as described. The embodiment(s) described, and references in the specification to “one embodiment,” “an embodiment,” “an example embodiment,” “some embodiments,” etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

1. A system for managing and diagnosing repairs of a consumer product comprising:
a mobile application executed on a mobile device having a processor, the mobile application comprising:
a scanning module for scanning an image of the consumer product by an end-user;
an image analysis module for extracting keypoints in the image; and
a repair application executed on a server, the repair application comprising:
an error detection module for determining a ranking of plurality of error messages based on the keypoints and the image,
wherein the error messages are stored in a product database associating each of the plurality of error message with a correction instruction; and
a transmission module for transmitting the plurality of error messages to the mobile application,
wherein the mobile application displays, to the end-user, a first error message from the plurality of error messages along with an associated correction instruction from the product database.

2. The system according to claim 1, wherein the consumer product is a medical device or a power tool.

3. The system according to claim 1, wherein the image analysis module denoises and grayscales the image prior to extracting keypoints.

4. The system according to claim 1, wherein ranking of the plurality of error messages is based on a number of results found in the product database.

5. The system according to claim 1, wherein the associated correction instruction is a video or a plurality of images arranged in a slideshow.

6. The system according to claim 1, wherein the repair application further comprises:
a product recommendation module for recommending a replacement product for the consumer product based on the first error message,
wherein the first error message indicates that the consumer product is not repairable.

7. The system according to claim 1, wherein the product recommendation module selects the replacement product from a plurality of replacement products based on the consumer product and details of the end-user.

8. The system according to claim 7, wherein the plurality of replacement products are stored in a product recommendation database associating a plurality of consumer products with the plurality of replacement products.

9. A system for managing repairs of a consumer product comprising:
a mobile application executed on a mobile device having a processor, the mobile application comprising:
a scanning module for scanning an image of the consumer product by an end-user;
an information acquisition module for collecting identifying information related to the image;
a code creation module for creating a unique machine-readable code including a unique identification number, the identifying information and a tracking number,
wherein an image of the unique machine-readable code is coupled to packaging housing the consumer product;
a transmission module for transmitting the unique machine-readable code to a repair system; and
a repair system for receiving the consumer product, the repair system comprising:
a receiving module for scanning the machine-readable code coupled to the packaging and extracting the unique identification number, the identifying information, and the tracking number; and
a repair module for matching the unique identification number to the received unique machine-readable code to verify receipt of the consumer product;
a technician module for assigning the consumer product to a technician for repairs; and
a return module for returning the repaired consumer product to the end-user.

10. The system according to claim 9, wherein the mobile application further comprises:
an information update system for receiving a plurality of repair milestones from the repair system,
wherein the information update system displays a plurality of milestone markers,
wherein each milestone marker is associated with a different step of the repair of the consumer product by the technician, and
wherein the milestone marker is changed from a first icon to a second icon upon completion of each step of the repair process associated with the milestone marker.

11. The system according to claim 10,
wherein the end-user can select a milestone marker from the plurality of milestone markers to display additional information.

12. The system according to claim 11, wherein in information update system displays the unique identification number in association with the plurality of milestone markers.

13. The system according to claim 9, wherein the repair system further comprises:
a product recommendation module for recommending a replacement product for the consumer product if a repair price is greater than the replacement product.

14. The system according to claim 13, wherein the replacement product is selected from a consumer product database associating consumer products with at least one replacement product.

15. The system according to claim 14, wherein a discount code is selected for the end-user for the replacement product based upon a current status of the end-user or the repair price.
